SEQUENCE LISTING (109 sequences)

SEQ ID NO: 1 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 2 - protein, 100 aa, Homo sapiens
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 3 - protein, 100 aa, Artificial Sequence
  FRB with T to L substitution at 2098
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 4 - protein, 100 aa, Artificial Sequence
  FRB segment of mTOR with T to H substitution at 2098 and to W at F at residue 2101
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 5 - protein, 100 aa, Artificial Sequence
  FRB segment of mTOR with K to P substitution at residue 2095
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 6 - protein, 100 aa, Artificial Sequence
  FRB sequence from SEQ ID NO. 1 protein of WO2016/135470
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEY

SEQ ID NO: 7 - protein, 107 aa, Artificial Sequence
  FKBP sequence from SEQ ID NO. 4 protein of WO2016/135470
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 8 - protein, 111 aa, Artificial Sequence
  Codon-wobbled FKBP sequence from SEQ ID NO. 4 protein of WO2016/135470 (FKBPw)
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLES

SEQ ID NO: 9 - protein, 286 aa, Artificial Sequence
  Amino acid numbers 266 to 551 of IL-2 receptor beta chain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 195 aa, Artificial Sequence
  IL7RA
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 11 - protein, 230 aa, Artificial Sequence
  IL9R
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 12 - protein, 569 aa, Artificial Sequence
  IL4RA
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 13 - protein, 437 aa, Artificial Sequence
  IL3RB
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 14 - protein, 189 aa, Artificial Sequence
  IL17RB
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 15 - protein, 91 aa, Artificial Sequence
  IL7RA 2Y truncated
KKRIKPIVWPSLPDHKKTLEHLCKKPRKNLNVSFNPESFLDCQIHRVDDIQARDEVEGFLQDTFPQQPILTSLGSNQEEAYVTMSSFYQNQ

SEQ ID NO: 16 - protein, 4 aa, Artificial Sequence
  STAT5 association motif
  misc_feature (2)..(3): Xaa can be any naturally occurring amino acid
  MISC_FEATURE (4)..(4): Xaa can be Phe or Leu
YXXX

SEQ ID NO: 17 - protein, 4 aa, Artificial Sequence
  STAT5 association motif
YCTF

SEQ ID NO: 18 - protein, 4 aa, Artificial Sequence
  STAT5 association motif
YFFF

SEQ ID NO: 19 - protein, 4 aa, Artificial Sequence
  STAT5 association motif
YLSL

SEQ ID NO: 20 - protein, 5 aa, Artificial Sequence
  STAT5 association motif
YLSLQ

SEQ ID NO: 21 - protein, 57 aa, Artificial Sequence
  JAK1 binding motif
KVLKCNTPDPSKFFSQLSSEHGGDVQKWLSSPFPSSSFSPGGLAPEISPLEVLERDK

SEQ ID NO: 22 - protein, 45 aa, Artificial Sequence
  JAK1 binding motif
NPWFQRAKMPRALDFSGHTHPVATFQPSRPESVNDLFLCPQKELT

SEQ ID NO: 23 - protein, 42 aa, Artificial Sequence
  JAK1 binding motif
GYICLRNSLPKVLNFHNFLAWPFPNLPPLEAMDMVEVIYINR

SEQ ID NO: 24 - protein, 42 aa, Artificial Sequence
  JAK1 binding motif
PLKEKSIILPKSLISVVRSATLETKPESKYVSLITSYQPFSL

SEQ ID NO: 25 - protein, 43 aa, Artificial Sequence
  JAK1 binding motif
RRRKKLPSVLLFKKPSPFIFISQRPSPETQDTIHPLDEEAFLK

SEQ ID NO: 26 - protein, 45 aa, Artificial Sequence
  JAK1 binding motif
YIHVGKEKHPANLILIYGNEFDKRFFVPAEKIVINFITLNISDDS

SEQ ID NO: 27 - protein, 41 aa, Artificial Sequence
  JAK1 binding motif
RYVTKPPAPPNSLNVQRVLTFQPLRFIQEHVLIPVFDLSGP

SEQ ID NO: 28 - protein, 43 aa, Artificial Sequence
  JAK2 binding motif
NYVFFPSLKPSSSIDEYFSEQPLKNLLLSTSEEQIEKCFIIEN

SEQ ID NO: 29 - protein, 49 aa, Artificial Sequence
  JAK2 binding motif
YWFHTPPSIPLQIEEYLKDPTQPILEALDKDSSPKDDVWDSVSIISFPE

SEQ ID NO: 30 - protein, 51 aa, Artificial Sequence
  JAK2 binding motif
YAFSPRNSLPQHLKEFLGHPHHNTLLFFSFPLSDENDVFDKLSVIAEDSES

SEQ ID NO: 31 - protein, 94 aa, Artificial Sequence
  IL2RB truncated - Y510
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 32 - protein, 208 aa, Artificial Sequence
  IL2RB truncated - Y510 & Y392
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 33 - protein, 52 aa, Artificial Sequence
  JAK3 motif
ERTMPRIPTLKNLEDLVTEYHGNFSAWSGVSKGLAESLQPDYSERLCLVSEI

SEQ ID NO: 34 - protein, 86 aa, Artificial Sequence
  JAK3 motif
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 35 - protein, 27 aa, Artificial Sequence
  CD28 Transmembrane domain
FWVLVVVGGVLACYSLLVTVAFIIFWV

SEQ ID NO: 36 - protein, 21 aa, Artificial Sequence
  CD8a TM domain
IYIWAPLAGTCGVLLLSLVIT

SEQ ID NO: 37 - protein, 81 aa, Artificial Sequence
  modified CD8a hinge plus CD8a TM domain for CAR
FVPVFLPAKPTTTPAPRPPTPAPTIASQPLSLRPEASRPAAGGAVHTRGLDFADIYIWAPLAGTCGVLLLSLVITLYCNHR

SEQ ID NO: 38 - protein, 54 aa, Artificial Sequence
  modified CD8a hinge
FVPVFLPAKPTTTPAPRPPTPAPTIASQPLSLRPEASRPAAGGAVHTRGLDFAD

SEQ ID NO: 39 - protein, 82 aa, Artificial Sequence
  wild type CD8a hinge
FVPVFLPAKPTTTPAPRPPTPAPTIASQPLSLRPEACRPAAGGAVHTRGLDFACDIYIWAPLAGTCGVLLLSLVITLYCNHR

SEQ ID NO: 40 - protein, 66 aa, Artificial Sequence
  CD28 hinge and transmembrane
IEVMYPPPYLDNEKSNGTIIHVKGKHLCPSPLFPGPSKPFWVLVVVGGVLACYSLLVTVAFIIFWV

SEQ ID NO: 41 - protein, 137 aa, Artificial Sequence
  RQR8
ACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 42 - protein, 11 aa, Artificial Sequence
  linker sequence
SGGGGSGGGGS

SEQ ID NO: 43 - protein, 10 aa, Artificial Sequence
  linker sequence
ETSGGGGSRL

SEQ ID NO: 44 - protein, 21 aa, Artificial Sequence
  linker sequence
SGGGGSGGGGSGGGGSGGGGS

SEQ ID NO: 45 - protein, 16 aa, Artificial Sequence
  linker sequence
SGGGGSGGGGSGGGGS

SEQ ID NO: 46 - protein, 5 aa, Artificial Sequence
  linker sequence
GGGGS

SEQ ID NO: 47 - protein, 4 aa, Artificial Sequence
  linker sequence
GGGS

SEQ ID NO: 48 - protein, 6 aa, Artificial Sequence
  linker sequence
GGGGGS

SEQ ID NO: 49 - protein, 7 aa, Artificial Sequence
  linker sequence
GGGGGGS

SEQ ID NO: 50 - protein, 6 aa, Artificial Sequence
  linker sequence
GGGGGG

SEQ ID NO: 51 - protein, 8 aa, Artificial Sequence
  linker sequence
GGGGGGGG

SEQ ID NO: 52 - protein, 18 aa, Artificial Sequence
  linker sequence
KESGSVSSEQLAQFRSLD

SEQ ID NO: 53 - protein, 14 aa, Artificial Sequence
  linker sequence
EGKSSGSGSESKST

SEQ ID NO: 54 - protein, 12 aa, Artificial Sequence
  linker sequence
GSAGSAAGSGEF

SEQ ID NO: 55 - protein, 16 aa, Artificial Sequence
  linker sequence
SGGGGSAGSAAGSGEF

SEQ ID NO: 56 - protein, 16 aa, Artificial Sequence
  linker sequence
SGGGLLLLLLLLGGGS

SEQ ID NO: 57 - protein, 16 aa, Artificial Sequence
  linker sequence
SGGGAAAAAAAAGGGS

SEQ ID NO: 58 - protein, 24 aa, Artificial Sequence
  linker sequence
SGGGAAAAAAAAAAAAAAAAGGGS

SEQ ID NO: 59 - protein, 24 aa, Artificial Sequence
  linker sequence
SGALGGLALAGLLLAGLGLGAAGS

SEQ ID NO: 60 - protein, 14 aa, Artificial Sequence
  linker sequence
SLSLSPGGGGGPAR

SEQ ID NO: 61 - protein, 25 aa, Artificial Sequence
  linker sequence
SLSLSPGGGGGPARSLSLSPGGGGG

SEQ ID NO: 62 - protein, 6 aa, Artificial Sequence
  linker sequence
GSSGSS

SEQ ID NO: 63 - protein, 7 aa, Artificial Sequence
  linker sequence
GSSSSSS

SEQ ID NO: 64 - protein, 6 aa, Artificial Sequence
  linker sequence
GGSSSS

SEQ ID NO: 65 - protein, 6 aa, Artificial Sequence
  linker sequence
GSSSSS

SEQ ID NO: 66 - protein, 6 aa, Artificial Sequence
  linker sequence
SGGGGS

SEQ ID NO: 67 - protein, 15 aa, Artificial Sequence
  linker sequence
GGGGSGGGGSGGGGS

SEQ ID NO: 68 - protein, 5 aa, Artificial Sequence
  linker sequence
GGGGG

SEQ ID NO: 69 - protein, 10 aa, Artificial Sequence
  linker sequence
GGGGSGGGGS

SEQ ID NO: 70 - protein, 20 aa, Artificial Sequence
  linker sequence
GGGGSGGGGSGGGGSGGGGS

SEQ ID NO: 71 - protein, 7 aa, Artificial Sequence
  linker sequence
GGGGGGG

SEQ ID NO: 72 - protein, 9 aa, Artificial Sequence
  linker sequence
GGGGGGGGG

SEQ ID NO: 73 - protein, 20 aa, Artificial Sequence
  signal peptide
MGTSLLCWMALCLLGADHAD

SEQ ID NO: 74 - protein, 19 aa, Artificial Sequence
  P2A peptide - cleavage domain
ATNFSLLKQAGDVEENPGP

SEQ ID NO: 75 - protein, 18 aa, Artificial Sequence
  T2A  peptide - cleavage domain
EGRGSLLTCGDVEENPGP

SEQ ID NO: 76 - protein, 20 aa, Artificial Sequence
  E2A peptide - cleavage domain
QCTNYALLKLAGDVESNPGP

SEQ ID NO: 77 - protein, 22 aa, Artificial Sequence
  F2A peptide - cleavage domain
VKQTLNFDLLKLAGDVESNPGP

SEQ ID NO: 78 - protein, 4 aa, Artificial Sequence
  Furin cleavage site
  misc_feature (2)..(3): Xaa can be any naturally occurring amino acid
RXXR

SEQ ID NO: 79 - protein, 4 aa, Artificial Sequence
  Furin cleavage site
RRKR

SEQ ID NO: 80 - protein, 286 aa, Artificial Sequence; identical to SEQ ID NO: 9
  IL2RB endodomain

SEQ ID NO: 81 - protein, 113 aa, Artificial Sequence
  CD3 zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 82 - protein, 41 aa, Artificial Sequence
  CD28 intracellular signalling
RSKRSRLLHSDYMNMTPRRPGPTRKHYQPYAPPRDFAAYRS

SEQ ID NO: 83 - protein, 48 aa, Artificial Sequence
  CD27 intracellular signalling
QRRKYRSNKGESPVEPAEPCHYSCPREEEGSTIPIQEDYRKPEPACSP

SEQ ID NO: 84 - protein, 4 aa, Artificial Sequence
  STAT3 signal
  misc_feature (2)..(3): Xaa can be any naturally occurring amino acid
YXXQ

SEQ ID NO: 85 - protein, 4 aa, Artificial Sequence
  STAT3 signal
YRHQ

SEQ ID NO: 86 - protein, 461 aa, Artificial Sequence
  Example construct (RQR8-frb-fkbp12-IL2RB truncated)
ACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVMASRILWHEMWHEGLEEASRLYFGERNVKGMFEVLEPLHAMMWEGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 87 - protein, 52 aa, Artificial Sequence
  JAK3 reverse orientation
IESVLCLRESYDPQLSEALGKSVGSWASFNGHYETVLDELNKLTPIRPMTRE

SEQ ID NO: 88 - protein, 92 aa, Artificial Sequence
  FKBP Sequence in SEQ ID NO. 1 of WO2016/135470
LEGVQVETISPGDGRTFPKRGQTCVVHYTGMLEDGKKFDSSRDRNKPFKFMLGKQEVIRGWEEGVAQMSVGQRAKLTISPDYAYGATGHPGI

SEQ ID NO: 89 - protein, 110 aa, Artificial Sequence
  FKBP Sequence in SEQ ID NO.2 of WO2016/135470
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 90 - protein, 521 aa, Homo sapiens
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 91 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 92 - protein, 260 aa, Nicotiana tabacum
MAVAPTAREENVYMAKLAEQAERYEEMVEFMEKVSNSLGSEELTVEERNLLSVAYKNVIGARRASWRIISSIEQKEESRGNEEHVNSIREYRSKIENELSKICDGILKLLDAKLIPSAASGDSKVFYLKMKGDYHRYLAEFKTGAERKEAAESTLTAYKAAQDIATTELAPTHPIRLGLALNFSVFYYEILNSPDRACNLAKQAFDEAIAELDTLGEESYKDSTLIMQLLRDNLTLWTSDMQDDGADEIKEDPKPDEAKN

SEQ ID NO: 93 - protein, 52 aa, Artificial Sequence
  C terminal 52 amino acids of Tobacco plasma membrane H+ ATPase (PMA2)
TNFNELNQLAEEAKRRAEIARQRELHTLKGHVESVVKLKGLDIETIQQSYTV

SEQ ID NO: 94 - protein, 521 aa, Artificial Sequence
  CNa12 GQ463594
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFEWSAPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 95 - protein, 521 aa, Artificial Sequence
  CNa22 GQ463595
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDRYNNKAAVLKYENNVMNIRQFNCSPHPFWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 96 - protein, 172 aa, Artificial Sequence
  CNb30 GQ463597
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNTKLADTQLQQIVDKTIINADKDGDGRISFEEFCAVVGGLDIHKKMVVDV

SEQ ID NO: 97 - protein, 521 aa, Artificial Sequence; identical to SEQ ID NO: 90
  CNa

SEQ ID NO: 98 - protein, 170 aa, Artificial Sequence
  CNb
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 99 - protein, 657 aa, Artificial Sequence
  pQTX-0353 (6aa linker)
MGTSLLCWMALCLLGADHADACPYSNPSLCETSGGGGSRLCPYSNPSLCSGGGGSMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVTRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 100 - protein, 668 aa, Artificial Sequence
  pQTX-0354 (17aa linker)
MGTSLLCWMALCLLGADHADACPYSNPSLCETSGGGGSRLCPYSNPSLCSGGGGSGGGGSGGGGSGMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVTRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 101 - protein, 94 aa, Artificial Sequence
  FRB 94 aa (DLL)
MILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 102 - protein, 94 aa, Artificial Sequence
  FRB 94aa (DLT)
MILWHEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 103 - protein, 99 aa, Artificial Sequence
  FRB 99 aa (DLL)
ASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEY

SEQ ID NO: 104 - protein, 719 aa, Artificial Sequence
  pQTX-0564
MAAPALSWRLPLLILLLPLATSWASAWSHPQFEKGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVTRRRKRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 105 - protein, 835 aa, Artificial Sequence
  pQTX-0565
MAAPALSWRLPLLILLLPLATSWASAWSHPQFEKMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVTRRRKRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 106 - protein, 835 aa, Artificial Sequence
  pQTX-0566
MAAPALSWRLPLLILLLPLATSWASAWSHPQFEKGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGGSGGGGSGGGGSGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVTRRRKRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 107 - protein, 495 aa, Artificial Sequence
  pQTX-0567
MLKPSLPFTSLLFLQLPLLGVGWSHPQFEKMILWHEMWHEGLEEASRLYFGERNVKGMFEVLEPLHAMMERGPQTLKETSFNQAYGRDLMEAQEWCRKYMKSGNVKDLLQAWDLYYHVFRRISKVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETTRRRKRGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 108 - protein, 624 aa, Artificial Sequence
  pQTX-0568
MLKPSLPFTSLLFLQLPLLGVGWSHPQFEKMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETTRRRKRGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 109 - protein, 624 aa, Artificial Sequence
  pQTX-0569
MLKPSLPFTSLLFLQLPLLGVGWSHPQFEKGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSMILWHEMWHEGLEEASRLYFGERNVKGMFEVLEPLHAMMERGPQTLKETSFNQAYGRDLMEAQEWCRKYMKSGNVKDLLQAWDLYYHVFRRISKSGGGGSGGGGSGGGGSGVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETTRRRKRGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2024/0052015 A1
(43) Pub. Date: Feb. 15, 2024

(54) INDUCIBLE SIGNALLING PROTEIN

(71) Applicant: QUELL THERAPEUTICS LIMITED, London (GB)

(72) Inventors: Marc MARTINEZ-LLORDELLA, London (GB); Luke HENRY, London (GB); Iain MCGILL, London (GB); Nathalie BELMONTE, London (GB)

(21) Appl. No.: 18/269,135

(22) PCT Filed: Dec. 22, 2021
(86) PCT No.: PCT/GB21/53414

§ 371 Date: Jun. 22, 2023

(30) Foreign Application Priority Data
Dec. 23, 2020 (GB) 2020541.5
Nov. 30, 2021 (GB) 2117281.2

(51) Int. Cl. C07K 14/715; C12N 9/90; C07K 14/705; A61K 39/00; C12N 9/12; C12N 9/16; C12N 5/0783

(52) U.S. Cl. CPC C07K 14/7155; C12N 9/90; C12Y 502/01008; C07K 14/70596; A61K 39/4611; A61K 39/4637; C12N 9/12; C12Y 207/10002; C12Y 301/03016; C12N 9/16; C12N 5/0637; A61K 2239/24; A61K 2239/22; C07K 2319/03; C07K 2319/02

(57) ABSTRACT

The present disclosure and invention relates to a chimeric protein useful in adoptive cell therapy (ACT) which is a membrane-associated signalling protein that can be induced to provide a cell expressing the protein with a STAT-5-mediated signal. Also provided are nucleic acid molecules encoding such a chimeric protein, 5 recombinant constructs, vectors and cells containing the nucleic acid molecule, methods of producing such cells, and therapeutic uses thereof.

### FIELD

The present disclosure and invention relate to a chimeric protein useful in adoptive cell therapy (ACT). In particular, the chimeric protein is a signalling protein which can be induced to provide a cell expressing the protein with a STAT-5-mediated signal. Also provided are nucleic acid molecules encoding such a chimeric protein, recombinant constructs, vectors and cells containing the nucleic acid molecule, methods of producing such cells, and therapeutic uses thereof.

### BACKGROUND

Adoptive cell therapy (ACT), that is the administration of functional immune cells to a subject, has become an established and evolving immunotherapeutic approach for various medical conditions, including notably malignant or infectious diseases. Tumour-infiltrating lymphocytes were initially shown to be effective in treating metastatic melanoma, and subsequently re-directed T-cells or NK cells expressing chimeric antigen receptors (CARs) or heterologous T-cell receptors (TCRs) to target different cellular target molecules have been developed and adopted for clinical use. Initial approaches used immune cells with cytotoxic properties, e.g. cytotoxic T-cells or NK cells, to target and kill unwanted or deleterious cells in the body, but more recently regulatory T cells (Tregs) have been developed for ACT. Tregs have immunosuppressive function. They act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation.

To be useful in ACT, the transplanted, or administered, cells need to survive and persist in the recipient (the subject of the ACT therapy) in a functional state long enough to exert a useful therapeutic effect. Further, to be prepared in sufficient numbers for therapeutic use, the cells need to be generated (e.g. engineered), cultured and expanded in vitro.

The growth factor interleukin-2 (IL-2) is essential for the homeostasis of immune cells, including notably Tregs (generation, proliferation, survival), as well as for their suppressive function and phenotypic stability. Activated conventional T cells (Tcons) are the main source of IL-2 in vivo. Tregs, in contrast, cannot produce IL-2 and depend on paracrine access to IL-2 produced by Tcons present in the microenvironment.

The availability of IL-2 has a critical impact on the therapeutic effects of Tregs expanded in vitro and transferred into patients. This is due to the following: 1) in vitro expansion protocols typically require high concentrations of IL-2, which renders Tregs highly dependent on this cytokine; 2) the concentration of IL-2 is often reduced in patients as a result of the administration of immunosuppressive drugs; and 3) within the inflamed tissue microenvironment access to IL-2 is often limited. Liver transplantation constitutes a particularly challenging indication, given that the levels of IL-2 in the inflamed liver are known to be reduced, which is further aggravated by the routine use of calcineurin inhibitors, which substantially decrease the capacity of Tcons to produce IL-2. The administration of low doses of exogenous IL-2 restores the Treg dysfunction induced by calcineurin inhibitors and promotes the accumulation of Tregs in the liver. However, a concern with the therapeutic use of low-dose IL-2 is the risk of simultaneously activating Tcons, which can enhance tissue damage.

In WO 2020/044055 an approach is described to circumvent the need to administer exogenous IL-2. In this case Treg cells are engineered to express a CAR which has been modified such that it is capable of providing a productive IL-2 signal to the cell upon binding to its target antigen. In other words, the intracellular signalling domain of the CAR, the endodomain, includes sequences, or domains, derived from IL receptors, which allow it to transmit an “IL-2 signal” in the absence of endogenous IL-2, and without the need for IL-2 binding. IL-2 signals through the transcription factor STAT5 (Signal Transducer and Activator of Transcription 5), which is phosphorylated in its active state by the kinases JAK1 and/or JAK2, which are normally activated when interleukins (e.g. IL-2) bind to their receptors. Accordingly, the CAR in WO 2020/044055 comprises an endodomain which comprises a STATS association motif and a JAK1- and/or a JAK2-binding motif.

Whilst WO 2020/04405 provides an important advance, there is a continuing need in the field of ACT for new and improved approaches, and in particular approaches which avoid or reduce the need to develop a modified CAR for each target, and which may have a more universal application.

### SUMMARY

The present inventors have developed a new signalling protein which can be used to impart an IL-2 signalling capacity to a cell, in the absence of exogenous IL-2. In particular, the signalling protein is inducible. That is, the signalling provided by the protein may be induced, when desired, by exposing a cell expressing the protein to an inducer molecule. The signalling protein is a chimeric protein comprising a binding domain, capable of interacting with (e.g. binding) an inducer molecule, and a signalling domain. The signalling domain is capable of transmitting a STAT-5 mediated signal when the chimeric protein is induced by interacting with (e.g. binding to) the inducer molecule. In particular, the chimeric protein is a dimerizable protein. The signalling activity of the chimeric protein is induced by dimerization, and dimerization of the protein is induced by the inducer. More particularly, the inducer may induce, or mediate, dimerization of the chimeric protein by binding to two separate sites in the binding domain, one in each of two copies of the protein. This brings two copies of the protein together and thereby allows two copies of the signalling domain or a portion of the signalling domain to dimerize, and thereby to be able to exert their function, namely transmission of a signal. In other words, dimerization of the binding domain, by interacting with, or binding to, the inducer, allows the functional protein component of the chimeric protein, here the signalling domain, to be dimerized in order to exert its function. The inducer may thus be seen as an inducer of dimerization, which allows a protein which is active in dimeric form to be dimerized, thereby to induce the activity of the protein. The inducer creates an interface between two binding sites, one in each copy of the protein, which in effect “dimerizes” the two binding sites.

As noted above, the STATS-mediated signal, which can be induced in a cell by interleukins such as IL-2, is a pro-survival signal, which helps the cell to survive and to maintain its ability to function during and after culture, and to persist and maintain its functional ability following administration to a subject in the course of therapy. It may alternatively be referred to as a persistence signal. Thus, the chimeric protein may be expressed in a cell to impart an inducible pro-survival signalling capacity to the cell. It has particular utility in cells prepared for use in ACT therapy, and may be expressed in such cells together with an antigen receptor, such as a TCR, or a CAR, or any chimeric receptor. The protein thus has utility in the engineering of cells for ACT.

Accordingly, a first aspect provides a chimeric protein capable of providing an inducible STATS-mediated signal in a cell expressing said protein, said protein comprising, linked together in any order:
(i) a first heterodimerization domain Ht1;
(ii) a second heterodimerization domain Ht2;
(iii) a signalling domain comprising a STAT5 association motif and a JAK1 and/or JAK2 binding motif, and optionally a JAK3 binding motif;
   wherein, in the presence of a chemical inducer of dimerization (CID), an identical pair of the chimeric proteins interact such that Ht1 from one chimeric protein heterodimerizes with Ht2 from the other chimeric protein, causing dimerization of the two signalling domains, such that upon said dimerization said STATS-mediated signal is induced.

The chimeric protein is a membrane-associated protein. More particularly, the chimeric protein is a membrane-bound protein. Accordingly, in an embodiment the chimeric protein may comprise a transmembrane domain, or a part thereof, or a domain, motif or group which allows association with the surface membrane of the cell, in other words, a membrane-targeting moiety. Such a moiety may be for example an amphipathic α-helix, a hydrophobic loop, or a lipid anchor, e.g. a myristate. It may be any moiety which is capable of associating with, e.g. binding to, or inserting into the cell membrane, or in any way associating with a component of a cell membrane (such components do not include a membrane-bound protein, but rather a component of the membrane bi-layer itself). In an embodiment, the chimeric protein comprises both transmembrane and extracellular domains.

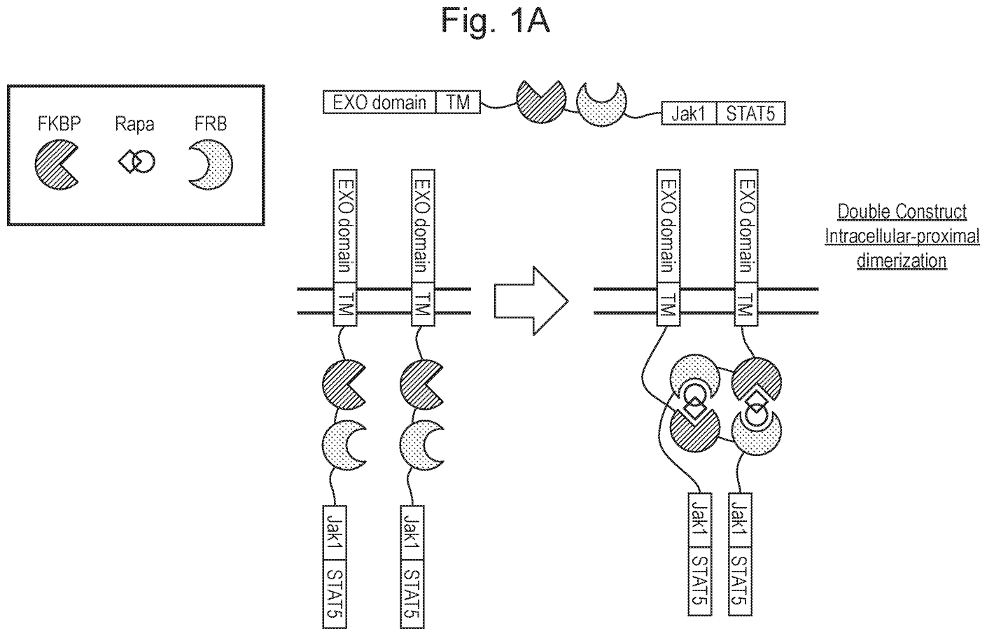

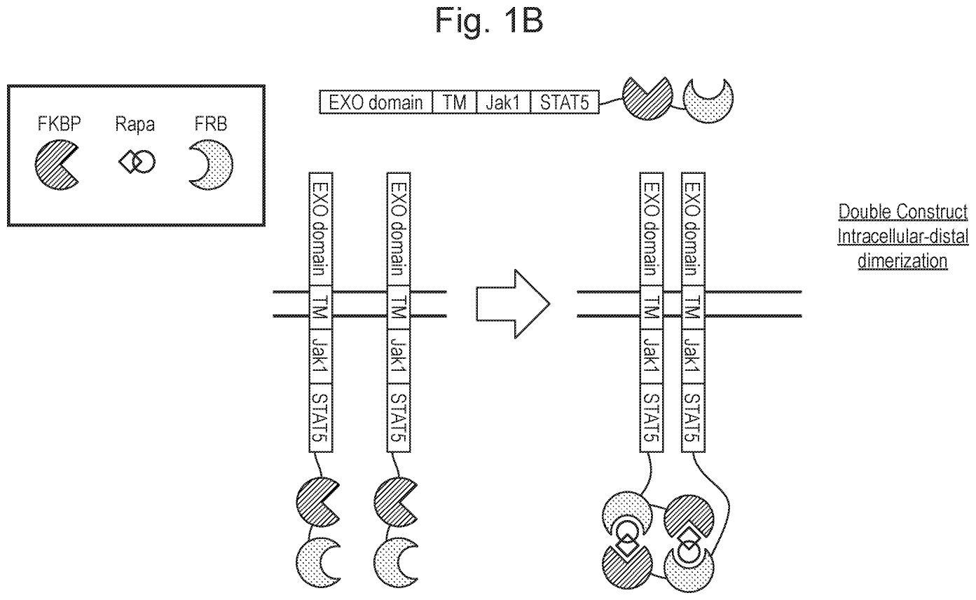

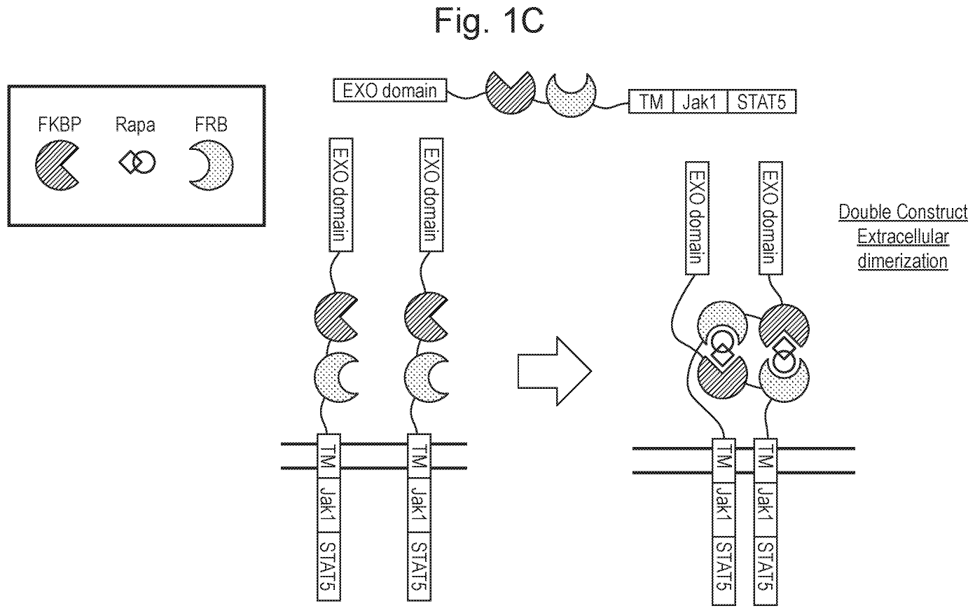

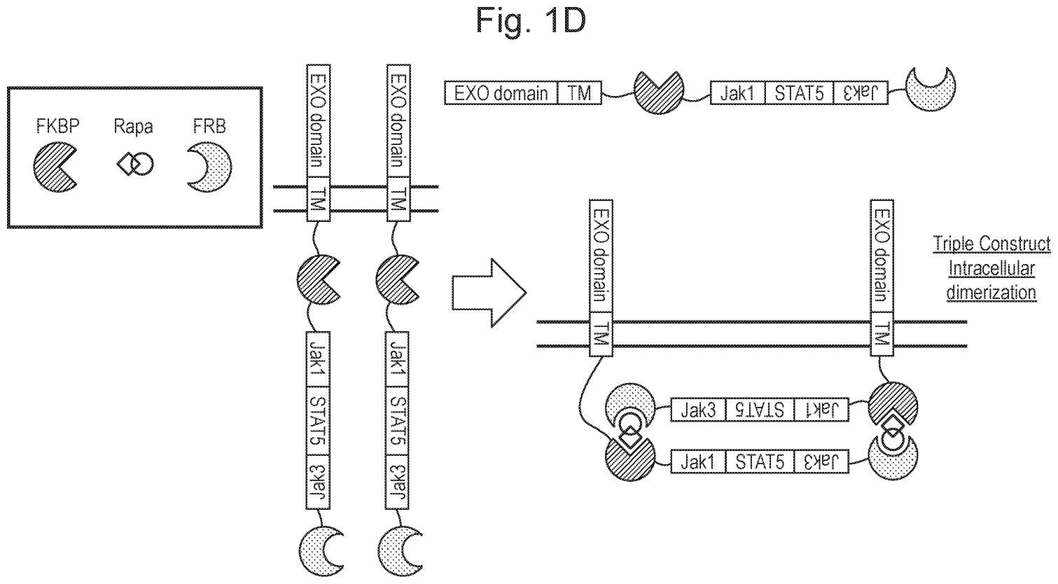

In an embodiment, the heterodimerization domains may bind to the CID, or more particularly they may comprise or constitute binding domains for the CID. In particular, the chimeric protein is configured such that a heterodimerization domain from each one of the pair of proteins binds to a CID molecule. The pair of proteins in a dimer is thus bound together via binding of their respective heterodimerization domains to 2 CID molecules, as depicted in FIG. 1.
It will be understood that the heterodimerization domains Ht1 and Ht2 are not identical (i.e. they are different). That is, the CID interacts with (e.g. binds to) two different binding sites, one in (or provided by) Ht1 and the other in (or provided by) Ht2.

The configuration of the chimeric protein is such that Ht1 does not heterodimerize to any significant extent with Ht2 within the same chimeric protein in the presence of the inducer.

In the chimeric protein, as discussed further below, Ht1 and Ht2 may be linked together. In other words, in such a protein Ht1 and Ht2 may be provided as a unit, or put another way, within a single inducer-binding domain. Such a single-unit inducer binding domain may be linked, or located, at the N- or the C-terminal of the signalling domain. Alternatively, Ht1 and Ht2 may be provided as separate binding sites in the chimeric protein. For example, they may each be linked, or located, at either end of the signalling domain. Thus, the inducer-binding domain may be provided in the chimeric protein in one or two parts. A single unit (1-part) binding domain comprises both Ht1 and Ht2. This allows “one-side” hetero-dimerization. A 2-part binding domain comprises two separate parts, which are spaced apart in the chimeric protein, the first part comprising one of Ht1 or Ht2 and the second part comprising the other of Ht1 and Ht2. This allows “two-side hetero-dimerization”.

Thus, various configurations of the chimeric protein are possible, wherein different components of the chimeric protein are linked together in various orders, as described further below.

The various components of the chimeric protein may be linked to each other directly or indirectly, as described further below. This includes the individual components of the binding domain (e.g. Ht1 and Ht2), and of the signalling domain. Thus, in the chimeric protein one or more linkers may be present, said linkers being between any two components of the chimeric protein. For example, there may be a linker between the binding domain, or a part thereof, and the signalling domain. Alternatively, or additionally there may be a linker between Ht1 and Ht2 (where Ht1 and Ht2 are present in a single unit, 1-part, binding domain), and/or there may be a linker between two or more components of the signalling domain.

A second aspect provides a nucleic acid molecule comprising a nucleotide sequence which encodes a chimeric protein as defined herein.

The nucleic acid molecule may be in the form of a construct, or more particularly, a recombinant construct, comprising the nucleic acid molecule and one or more other nucleotide sequences (a nucleotide sequence of interest). For example, the construct may comprise the nucleic acid molecule and a regulatory sequence, e.g. an expression control sequence, and/or a sequence encoding another functional protein (or more generally, a protein of interest), for example a receptor, e.g. a CAR or TCR etc. The construct may comprise one or more co-expression sequences linking the nucleic acid molecule with one or more other coding nucleotide sequences.

A third aspect provides a vector comprising a nucleic acid molecule or construct as defined herein.

The vector may be a viral or non-viral vector. In an embodiment the vector may comprise a nucleic acid molecule as defined herein and a further nucleotide sequence encoding a protein of interest, notably a receptor, e.g., a CAR or TCR.

A fourth aspect provides a cell which expresses a chimeric protein as defined herein, or which comprises a nucleic acid molecule, construct or vector as defined herein.

The cell may express the chimeric protein on the cell surface or in proximity to the cell membrane. Thus, the chimeric protein may be expressed on a cell or within a cell, associated with the cell membrane.

Also provided according to this aspect is a cell population comprising a cell as defined herein.

In an embodiment, the cell is an immune cell, or a precursor thereof. The cell may be a stem cell, or more particularly a haemopoietic stem cell (HSC) or pluripotent stem cell (PSC), e.g. an induced pluripotent stem cell (iPSC). Particularly, the cell may be a lymphocyte, or more particularly a T-cell, NK cell, dendritic cell or myeloid-derived suppressor cell (MDSC). In an embodiment, the T-cell may be a Treg cell. The cell may be a primary cell or from a cell line. In another embodiment the cell may be a production host cell, that is a cell into which the nucleic acid molecule construct or vector is introduced in order to produce the chimeric protein, or a viral vector encoding the chimeric protein.

A fifth aspect provides a method of preparing a cell as defined herein (i.e. a cell according to the fourth aspect), said method comprising introducing into a cell (e.g. transducing or transfecting a cell with), a nucleic acid molecule, construct or vector as defined herein. The method may include allowing the chimeric protein to be expressed by the cell. This may include, for example, culturing the cell.

Such a method may further comprise a preceding step of isolating, enriching providing or generating a cell to be used in the method. Further, a cell may be isolated or enriched or generated after the step of introducing the nucleic acid molecule. For example, the nucleic acid molecule may be introduced into a precursor or progenitor cell, e.g. a stem cell, and the cell may then be induced or caused to differentiate, or change, into a desired cell type. For example, an iPSC cell may be differentiated into an immune effector cell (e.g. a Treg or other T cell) or a Tcon cell may be converted into a Treg cell, etc.

This aspect may also include a method of preparing a chimeric protein as defined herein, said method comprising introducing into a cell, a nucleic acid molecule, construct or vector as defined herein, allowing the chimeric protein to be expressed by the cell, and optionally detecting the chimeric protein.

A sixth aspect provides a method of promoting the survival or persistence of a cell, said method comprising introducing into the cell, a nucleic acid molecule, construct or vector as defined herein, and exposing the cell to (or contacting the cell with) a CID. The CID induces dimerization, and signalling by the chimeric protein, and provides a pro-survival signal to the cell.

This aspect may comprise administering a cell as defined herein to a subject, and administering a CID to the subject. The CID may be administered before, during or after administration of the cell. Thus, in this aspect, the method may be carried out in vivo. Alternatively, the method of the sixth aspect may be carried out in vitro/ex vivo.

As noted above, the chimeric protein may advantageously be expressed by a cell in the context of therapy. Whilst the cell may be an unmodified cell, in the sense of not being further genetically engineered for therapeutic use, for example a T cell isolated from a subject, or a cell derived from such an isolated cell (although of course the cell would be modified by the present method to express the chimeric protein), typically the cell will be a cell which is additionally modified, or engineered to express a further molecule (i.e. a further protein), notably a receptor, e.g. a CAR or TCR.

Thus, a seventh aspect provides a method of preparing a cell for use in adoptive cell transfer therapy (ACT), said method comprising providing said cell with a chimeric protein as defined herein. More particularly, this method may comprise introducing into said cell a nucleic acid molecule, construct or vector as defined herein. The method may also comprise introducing into the cell a separate nucleic acid molecule, construct or vector which comprises a nucleotide sequence which encodes a therapeutic protein, notably a receptor, e.g. a CAR or TCR.

An eighth aspect provides a pharmaceutical composition comprising a cell, cell population or a vector as defined herein, together with at least one pharmaceutically acceptable carrier or excipient. In an embodiment the cell expresses, or the vector comprises an additional nucleotide sequence encoding a further protein, notably a receptor, e.g. a CAR or TCR. In another embodiment the cell comprises a separate nucleic acid molecule, construct or vector which comprises a nucleotide sequence which encodes a further protein, notably a receptor, e.g. a CAR or TCR.

A ninth aspect provides a cell or cell population as defined herein, or a pharmaceutical composition of as defined herein, or a vector as defined herein for use in therapy. Particularly, the cell, cell population or a pharmaceutical composition comprising the cell or cell population may be for ACT. The vector or pharmaceutical composition comprising the vector may be for gene therapy. The ACT or gene therapy may be for the treatment or prevention of any condition which is responsive to ACT or gene therapy, in particular immunotherapy by ACT or gene therapy.

A tenth aspect provides a cell, cell population, vector or pharmaceutical composition as defined herein for use in the treatment of or prevention of cancer, or an infectious, neurodegenerative, inflammatory, autoimmune or allergic disease or any condition associated with an unwanted or deleterious immune response. In particular, where the cell is a Treg or other immunosuppressive cell, the cell may be used for inducing immunosuppression (i.e. for suppressing an unwanted or deleterious immune response), for example to improve and/or prevent immune-mediated organ damage in inflammatory disorders, autoimmune or allergic diseases or conditions, and in transplantation.

This aspect also provides a method of adoptive cell transfer therapy, said method comprising administering to a subject in need of said therapy a cell, cell population, or pharmaceutical composition as defined herein, particularly an effective amount of said cell, cell population or pharmaceutical composition.

Also provided is a method of treating or preventing cancer, or an infectious, neurodegenerative, inflammatory, autoimmune or allergic disease or a condition associated with an unwanted or deleterious immune response, said method comprising administering to a subject in need thereof a cell, cell population, vector or pharmaceutical composition as defined herein, particularly an effective amount of said cell, cell population, vector or pharmaceutical composition.

Further, there is provided use of a cell, cell population or vector as defined herein in the manufacture of a medicament for use in treating or preventing cancer, or an infectious, neurodegenerative, inflammatory, autoimmune or allergic disease or a condition associated with an unwanted or deleterious immune response.

In some embodiments of these therapeutic aspects the use may be in induction of tolerance to a transplant; treating and/or preventing cellular and/or humoral transplant rejection; treating and/or preventing graft-versus-host disease (GvHD), an autoimmune or allergic disease; to promote tissue repair and/or tissue regeneration; or to ameliorate inflammation. In particular, in such embodiments the cell may be a Treg cell.

In the various therapeutic aspects set out above the cell, cell population, vector or pharmaceutical composition may be for use in combination with, or together with, a CID.

Accordingly, an eleventh aspect provides a combination product comprising (a) a cell, cell population, vector or pharmaceutical composition as defined herein, and (b) a CID, for use in therapy, particularly ACT or gene therapy. The therapy may be any therapy as defined above, and further described herein.

The components (a) and (b) of the combination product may be for separate, sequential or simultaneous use.

DESCRIPTION OF THE FIGURES

FIG. 1 depicts various formats of the chimeric protein wherein Ht1 is FKBP, Ht2 is FRB, the CID is rapamycin, TM is a transmembrane domain, Exo is the extracellular domain, and the signalling domain comprises, N-terminal to C-terminal, JAK1-STAT5 motifs (double construct) or JAK1-STAT5-JAK3 motifs (triple construct; JAK3 is reverse orientation), wherein:
in (A), the chimeric protein is a membrane protein with an exo domain, comprising a 1-part inducer binding domain linked to the N-terminus of the signalling domain (double construct/intracellular proximal one-sided dimerization);
in (B), the chimeric protein is a membrane protein with an exo domain, comprising a 1-part inducer binding domain linked to the C-terminus of the signalling domain (double construct/intracellular distal one-sided dimerization);
in (C), the chimeric protein is a membrane protein with an exo domain, comprising a 1-part inducer binding domain linked to the C-terminus of the Exo domain (double construct/extracellular one-sided dimerization);
in (D), the chimeric protein is a membrane protein with an exo domain comprising a 2 part inducer-binding domain wherein FKBP is linked to the C-terminal of the TM domain and to the N-terminal of the signalling domain and FRB is linked to the C-terminal of the signalling domain (triple construct/two-sided intracellular dimerization).

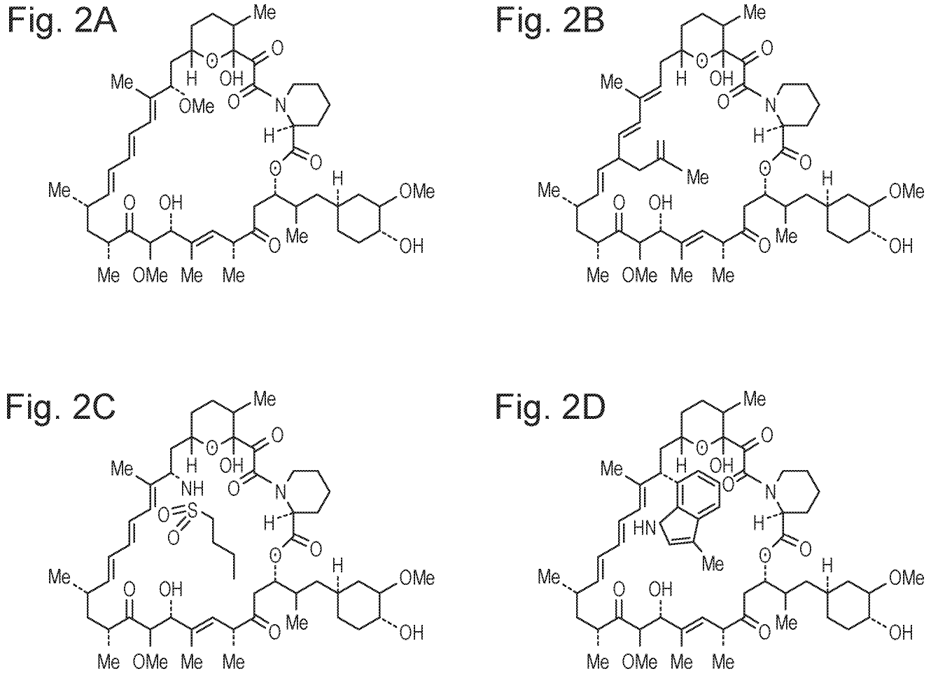

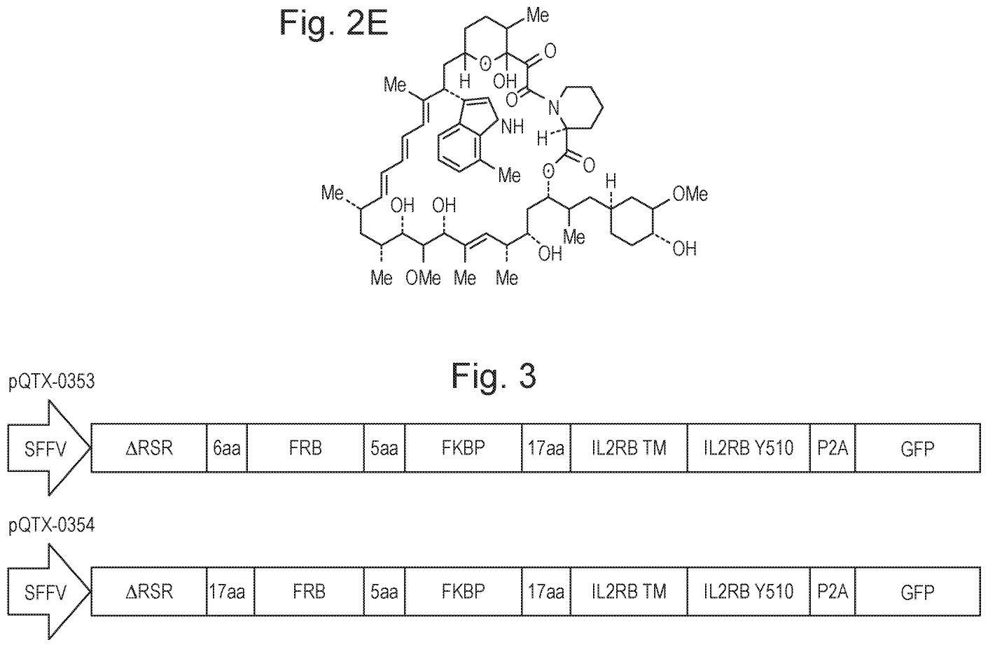

FIG. 2 shows Rapamycin and rapalogs. A) Rapamycin; B) C-20-methyllyrlrapamycin (MaRap); C) C16(S)-Butylsulfonamidorapamycin (C16-BS-Rap); D) C16-(S)-3-mehylindolerapamycin (C16-iRap); and E) C16-(S)-7-methylindolerapamycin (AP21976/C16-AiRap).

FIG. 3 shows schematic diagrams of the constructs, pQTX-0353 (SEQ ID NO. 99) and pQTX-0354 (SEQ ID NO. 100). Both constructs contain the SFFV (Spleen focus forming virus) promoter followed by a truncated safety switch (ARSR8); FRB (FKBP Rapamycin Binding); 6aa or 17aa linker (6aa in pQTX-0353 (SEQ ID NO. 99) and 17aa in pQTX-0354 (SEQ ID NO. 100)); 5aa linker; FKBP; 17aa linker; IL2RB transmembrane domain; truncated IL2RB signaling domain containing the Y510; P2A self-cleaving peptide; eGFP reporter gene.

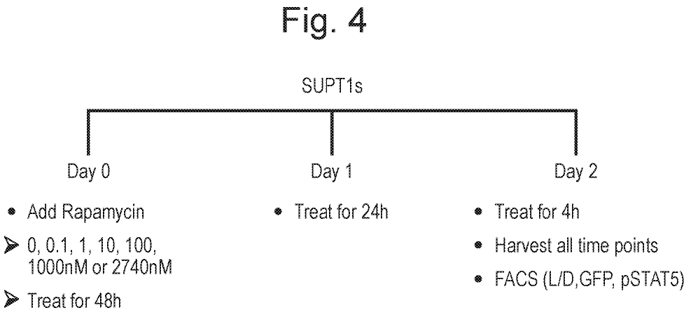

FIG. 4 shows a schematic of the experimental set up for a pSTAT5 assay using SupT1 cells.

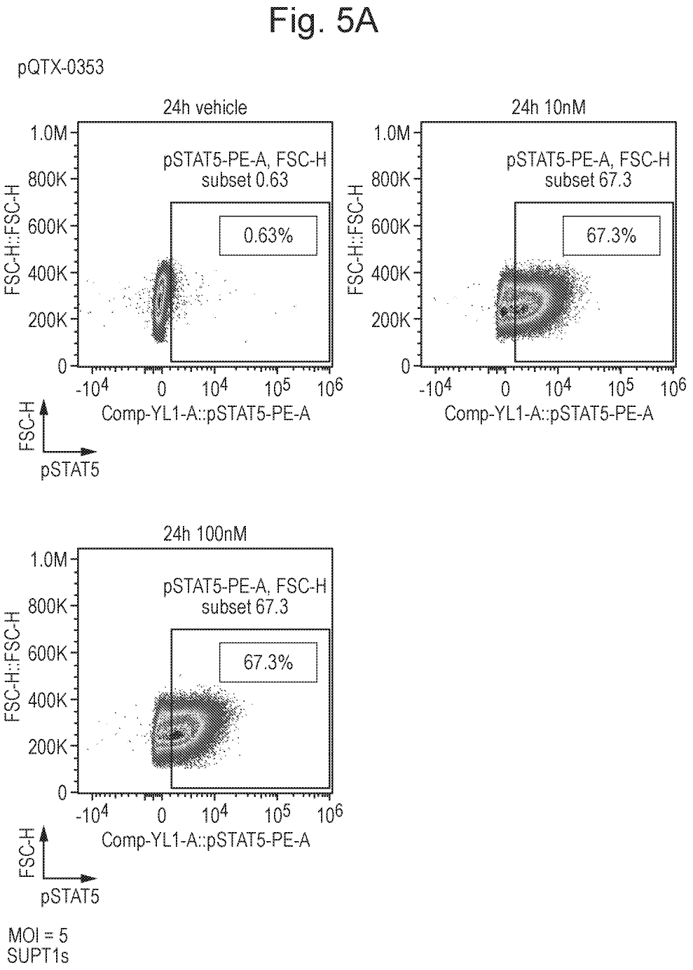

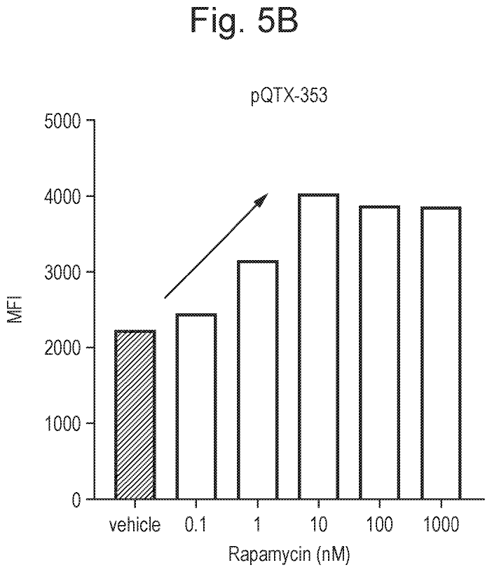

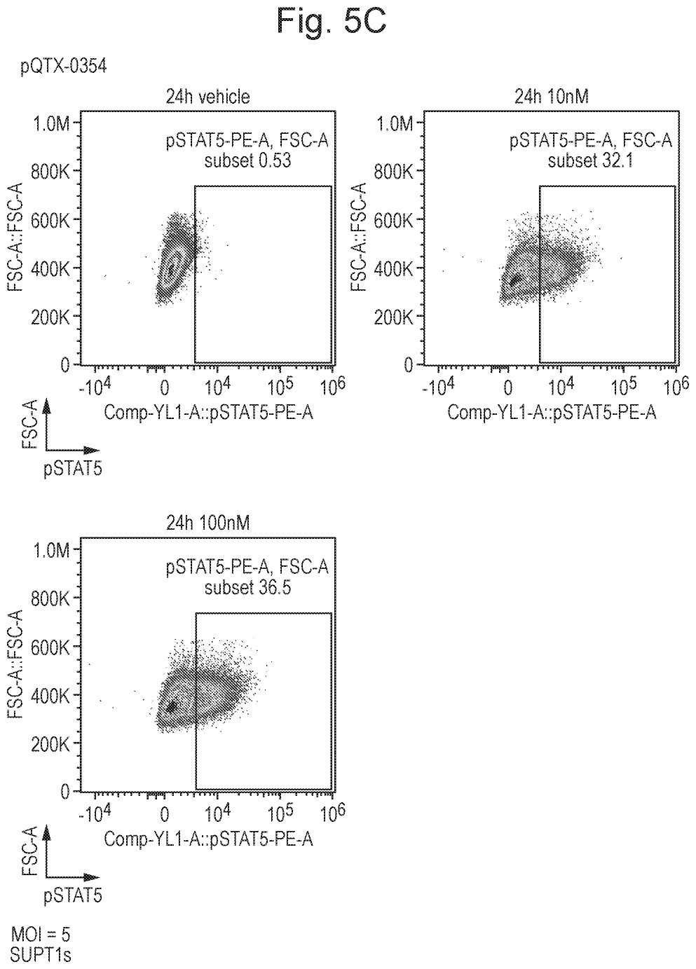

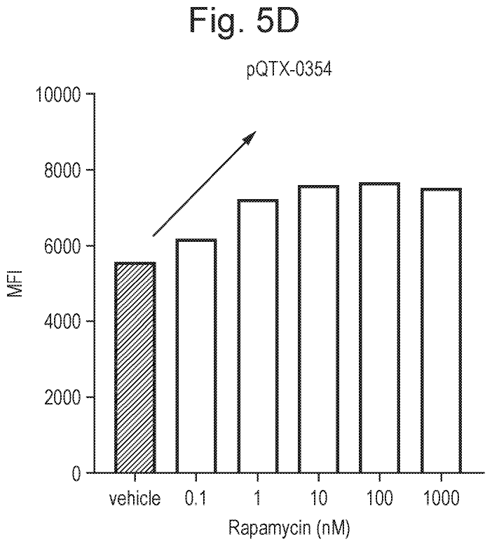

FIG. 5 shows that SupT1 cells transduced with pQTX-0353 (SEQ ID NO. 99) and pQTX-0354 (SEQ ID NO. 100) demonstrate a significant increase in pStat5 expression upon Rapamycin treatment. Gating strategy: Cells were gated for, followed by a singlet gate, followed by a live/dead gate, followed by gating on the transduced population of cells (GFP+ve). These cells were then analysed for pSTAT5-PE signal. FIG. 5A shows SupT1 cells transduced with pQTX-0353 (SEQ ID NO. 99) and stained for pSTAT5 expression. The FACS plots displayed show cells at 24 hours post Rapamycin treatment with either no Rapamycin, 10nM or 100nM of rapamycin. The percentage of cells positive for pSTAT5 is displayed. The graph shows the geometric mean fluorescence (MFI) of the pSTAT5 signal from the stained SupT1 transduced with pQTX-0353 (SEQ ID NO. 99) cells, showing the fluorescence intensity at all concentrations of Rapamycin tested (0, 0.1 nM, 1 nM, 10 nM, 100 nM and 1000 nM). FIG. 5B shows SupT1 cells transduced with pQTX-0354 (SEQ ID NO. 100) and stained for pSTAT5 expression. The FACS plots displayed show cells at 24 hours post Rapamycin treatment with either no Rapamycin, 10 nM or 100 nM of rapamycin. The percentage of cells positive for pSTAT5 is displayed. The graph shows the geometric mean fluorescence (MFI) of the pSTAT5 signal from the stained SupT1 transduced with pQTX-0354 (SEQ ID NO. 100) cells, showing the fluorescence intensity at all concentrations of Rapamycin tested (0, 0.1 nM, 1 nM, 10 nM, 100 nM and 1000 nM).

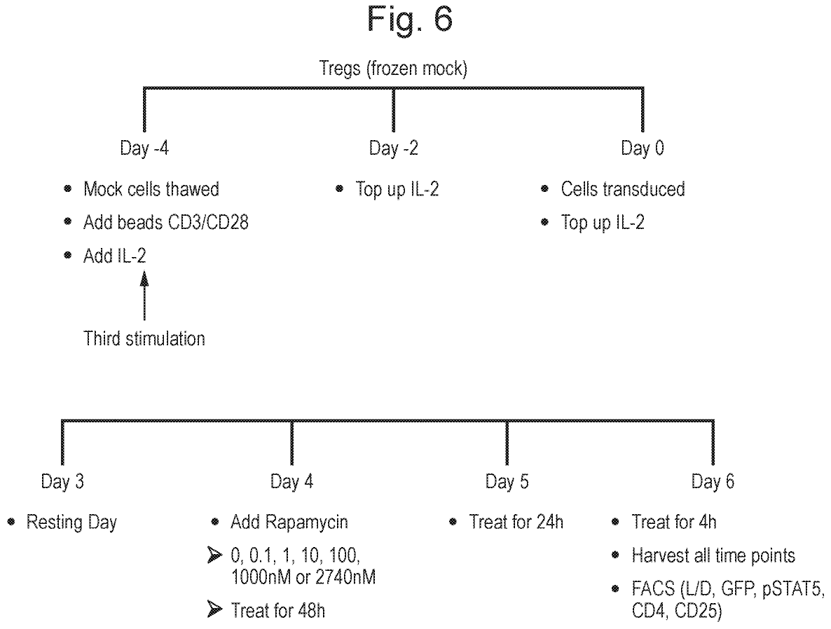

FIG. 6 shows a schematic of the experimental set up for a pSTAT5 assay using Treg cells.

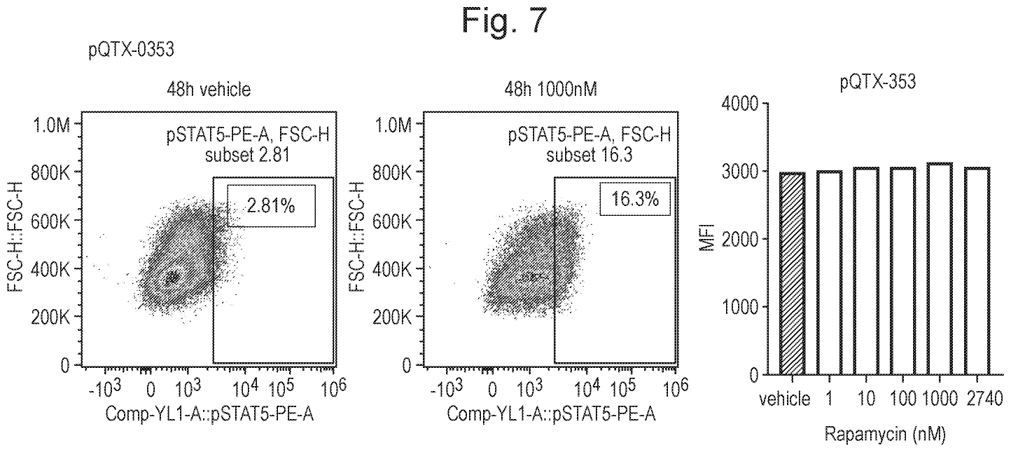

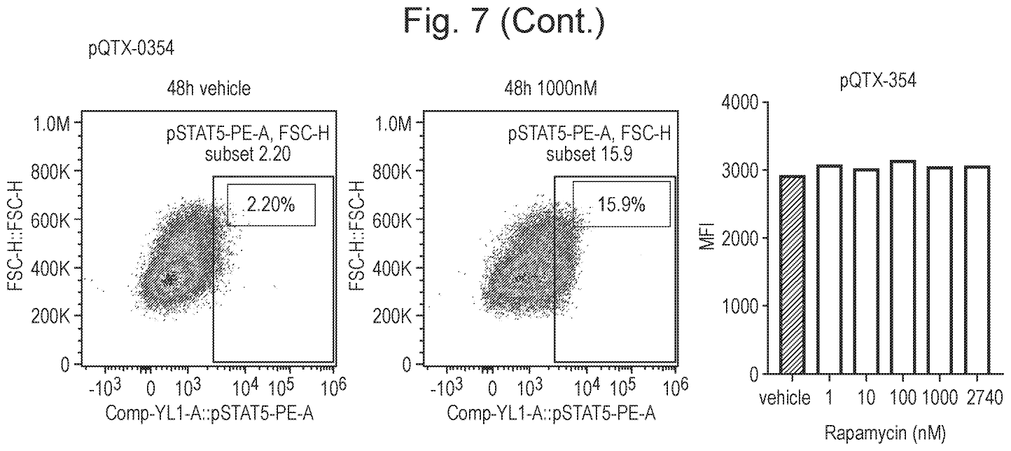

FIG. 7 shows that human Tregs transduced with (A) pQTX-0353 (SEQ ID 20 NO. 99) or (B) pQTX-0354 (SEQ ID NO. 100) demonstrate an increased pSTAT5 expression level upon Rapamycin treatment. pQTX-0353 (SEQ ID NO. 99) transduced human Tregs (MOI 5) or pQTX-0354 (SEQ ID NO. 100) transduced human Tregs (MOI 2) were rested for 24 hours prior to the assays, cell was then treated with 0 nM, 1 nM, 10 nM, 100 nM, 1000 nM or 2740 nM of rapamycin for 48, 24 or 4 hours prior to staining. FACS plots show GFP+cell populations and the pSTAT5 expression levels after 48 hour treatment of rapamycin with vehicle alone or with 1000 nM or 100 nM for pQTX-0353 (SEQ ID NO. 99) or pQTX-0354 (SEQ ID NO. 100), respectively. Graphs show the mean fluorescence intensity (MFI) of pSTAT5 expression levels with pQTX-0353 (SEQ ID NO. 99) or pQTX-0354 (SEQ ID NO. 100) with the various indicated rapamycin concentrations treatments.

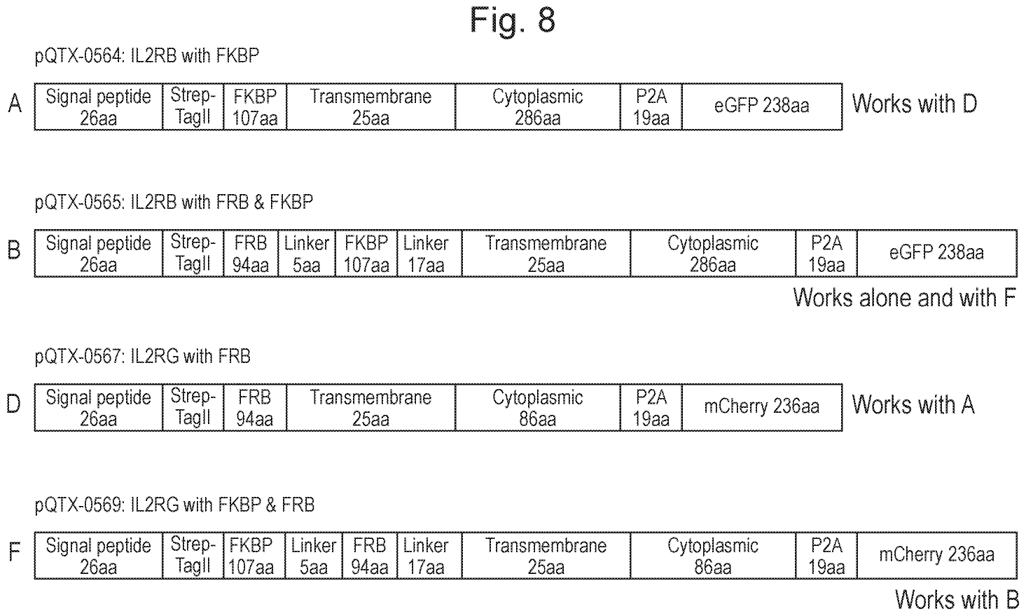

FIG. 8 shows schematic diagrams of additional membrane-bound constructs, pQTX-0564, pQTX-0565, pQTX-0567 and pQTX-0569 (SEQ ID NOs. 104-109, respectively). All constructs contain a Strep tag followed by: pQTX-0564: FKBP; Transmembrane domain; Cytoplasmic domain; P2A self-cleaving peptide; eGFP reporter gene. pQTX-0565: FRB (FKBP Rapamycin Binding); 5aa linker; FKBP; 17aa linker; Transmembrane domain; cytoplasmic domain; P2A self-cleaving peptide; eGFP. pQTX-0567: FRB (FKBP Rapamycin Binding); Transmembrane domain; Cytoplasmic domain; P2A self-cleaving peptide; mCherry reporter gene. pQTX-0569: FKBP; 5aa linker; FRB (FKBP Rapamycin Binding); 17aa linker; Transmembrane domain; Cytoplasmic domain; P2A self-cleaving peptide; mCherry reporter gene.

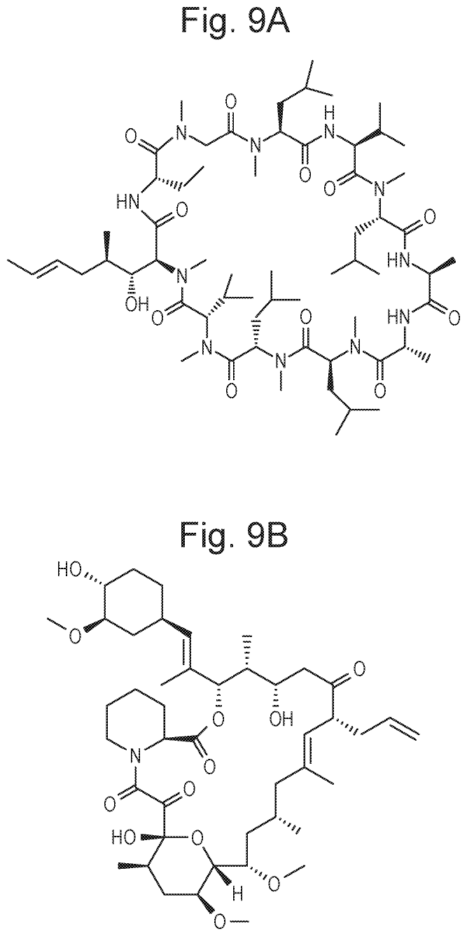

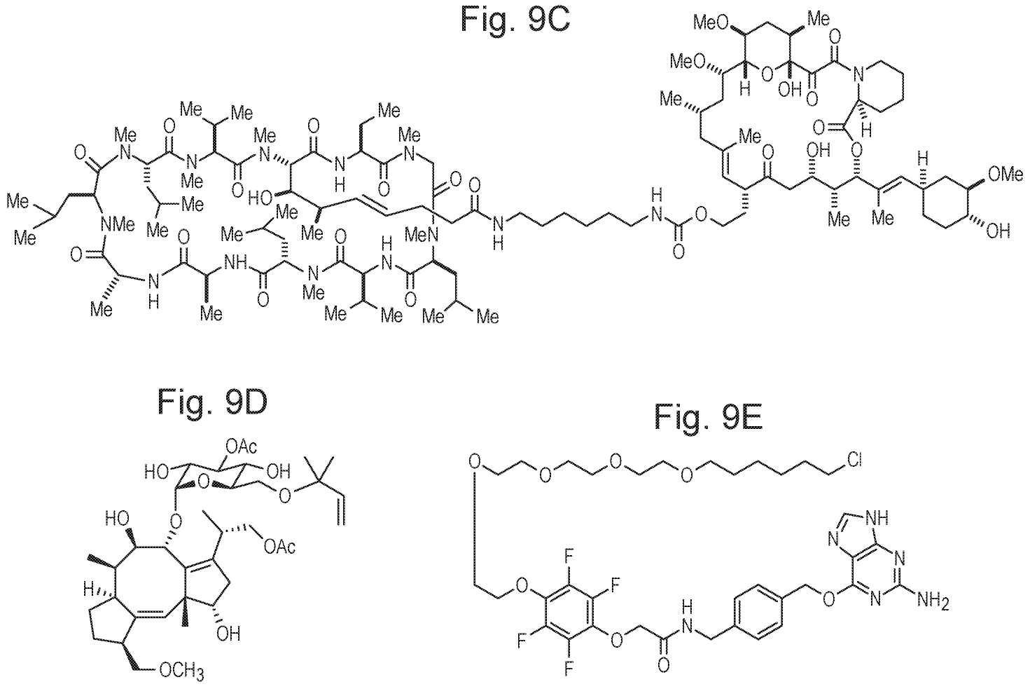

FIG. 9 shows the structures of CID molecules (A) cyclosporine (CsA); (B) FK506; (C) FKCsA; (D) Fusicoccin and (E) HaSX8.

### DETAILED DESCRIPTION

The subject of the products, methods and uses herein is a chimeric signalling protein which can be induced to promote the survival, or indeed the functionality, of a cell by which it is expressed. The protein thus has utility in adoptive cell transfer, to assist in the preparation of cells for ACT, and/or to help keep the cells alive and functional following transfer to a subject.

The chimeric protein is based on a dimerization system, in which a pair of cognate hetero-dimerizable domains are comprised within the protein, and in the presence of an inducer, a so-called chemical inducer of dimerization (CID), the cognate heterodimerization domains of two protein molecules are brought together to form a hetero-dimer. This brings together the signalling domains of two proteins, allowing them to dimerize and become functional. Thus, a feature of the present chimeric protein is that the binding domain and the signalling domain are present in the same protein chain. In other words, the chimeric protein monomer is a single chain protein.

The heterodimerization between the heterodimerization domains Ht1 and Ht2 includes any means or method by which Ht1 and Ht2 are associated together (it will be understood that this means associated together in the presence of the CID). This can be a direct or indirect association, and does not mean that the two domains need to be bind to one another, although this is not excluded. In an embodiment, the two interacting domains each bind to an inducer, the CID. The inducer thus creates an interface between Ht1 and Ht2 and brings them together as a dimer.

Such chemically-induced heterodimerization systems are known in the art, using various CID molecules, and different protein domains for heterodimerization, and are described further below.

The binding domain of the chimeric protein, which comprises Ht1 and Ht2, which as noted above may be in 1- or 2-part form, thus allows two chimeric protein molecules to interact with an inducer molecule (CID). It may thus alternatively be referred to as an interacting domain. Ht1 in one chimeric protein molecule binds to an inducer molecule which is also bound by Ht2 from another chimeric protein molecule, and in this way the Ht1 and Ht2 in the 2 chimeric protein molecules become dimerized, particularly heterodimerized, and therefore the chimeric protein is also dimerized.

Since each monomer of the chimeric protein comprises two heterodimerization domains Ht1 and Ht2, each protein pair will bind to two molecules of the CID. Each CID molecule is bound by Ht1 from one protein and Ht2 from the other.

As further noted above, the configuration of the chimeric protein is such that Ht1 does not significantly heterodimerize with Ht2 within the same chimeric protein molecule. However, when two chimeric proteins come together in the presence of a chemical inducer of dimerization (CID) Ht1 from one chimeric protein heterodimerizes with Ht2 from the other chimeric protein, causing dimerization of the two signaling domains.

For example, in a cell expressing the present chimeric protein the presence of the CID causes a greater proportion of dimerization between two chimeric proteins, than heterodimerization within the same chimeric protein. The amount of chimeric proteins which are heterodimerized within the same molecule in a cell or cell population, or in solution, may be less than 50%, 40%, 30%, 20%, 10%, 5% or 1% of the amount of chimeric proteins which are heterodimerized with a separate chimeric protein molecule, in the presence of the CID.

The concept of chemically induced dimerization mediated by small molecule inducers has been known for many years, and has been used as a tool to control dimerization between proteins of interest that are fused to inducer-binding domains. Such systems have been described for use in cell biology for different applications, to bring proteins into proximity, for example to investigate signalling pathways and other biological mechanisms, in medicine to degrade or inactivate pathogenic proteins, and in gene and cell therapy. A typical CID has the feature of being able to interact with, or bind to, two proteins or protein domains, one on either side of the molecule. It thus has two binding sites, or binding surfaces (or more generally, interaction sites). In the case of heterodimerization, the CID is capable of interacting with, or binding to, two different proteins or protein domains. The original systems were based on the macrolides FK506 and rapamycin, which are capable of binding to, and therefore inducing heterodimerization of, various different proteins or protein domains, including FK506-binding protein (FKBP), the FKBP-rapamycin domain of mTOR (FRB), calcineurin, and cyclophilin, which can be used in different combinations to achieve heterodimerization domain pairs and CID combinations, as detailed below. Such systems may include the use of cyclosporine, which binds to calcineurin or to cyclophilin. Subsequently, other CID heterodimerization systems based on different molecules have been developed and are described in the literature.

In an embodiment the CID is rapamycin or an analogue thereof, and Ht1 and Ht2 are protein domains which bind thereto. Rapamycin and rapamycin analogues induce heterodimerization by generating an interface between the FRB domain of mTOR and a FK506-bindng protein (FKBP). This association results in FKBP blocking access to the mTOR active site inhibiting its function. While mTOR is a very large protein, the precise small segment of mTOR required for interaction with Rapamycin is known and can be used.

The macrolides rapamycin and FK506 act by inducing the heterodimerization of cellular proteins. Each drug binds with a high affinity to the FKBP12 protein, creating a drug-protein complex that subsequently binds and inactivates mTOR/FRAP and calcineurin, respectively. The FKBP-rapamycin binding (FRB) domain of mTOR has been defined and applied as an isolated 89 amino acid protein moiety that can be fused to a protein of interest. Rapamycin can then induce the approximation of FRB fusions to FKBP12 or proteins fused with FKBP12.

In the present context one of the heterodimerization domains (Ht1 or Ht2) may be or comprise FRB, and the other heterodimerization domain (Ht2 or Ht1) may be or comprise FKBP. The terms “FRB” and “FKBP” include variants thereof. Such variants may include amino acid sequences having one or more amino acid modifications (e.g. substitutions, additions and/or deletions) relative to the native sequence. The term “FKBP” includes FKBP12.

Rapamycin has several properties of an ideal dimerizer: it has a high affinity (KD<1 nM) for FRB when bound to FKBP, and is highly specific for the FRB domain of mTOR. Rapamycin is an effective therapeutic immunosuppressant with a favourable pharmacokinetic and pharmacodynamics profile in mammals. Pharmacological analogues of Rapamycin with different pharmacokinetic and dynamic properties such as Everolimus, Temsirolimus and Deforolimus (Benjamin et al, Nature Reviews, Drug Discovery, 2011) may also be used according to the clinical setting.

In order to prevent rapamycin binding and inactivating endogenous mTOR, the surface of rapamycin which contacts FRB may be modified. Compensatory mutation of the FRB domain to form a surface that accommodates the “bumped” rapamycin restores dimerizing interactions only with the FRB mutant and not to the endogenous mTOR protein.

Bayle et al. (Chem Bio; 2006; 13; 99-107) describes various rapamycin analogues, or “rapalogs” and their corresponding modified FRB binding domains. For example, Bayle et al. (2006) describes the rapalogs: C-20-methyllyrlrapamycin (MaRap), 016(S)-Butylsulfonamidorapamycin (C16-BS-Rap) and C16-(S)-7-methylindolerapamycin (AP21976/C16-AiRap), as shown in FIG. 2, in combination with the respective complementary binding domains for each. Other rapamycins/rapalogs include sirolimus and tacrolimus (FK506).

Thus, in such an embodiment, one of Ht1 and Ht2 comprises FKBP and the other comprises FRB. In particular, the FKBP domain may comprise FKBP12.

Heterodimerization between the FRB domain of one copy of the chimeric protein molecule and the FKBP domain of another copy of the molecule causes dimerization of the signalling domains.

Rapamycin is a standard pharmaceutical with well understood properties, excellent bioavailability and volume of distribution and which is widely available. Rapamycin also does not aggravate the condition being treated, in fact, as it is an immunosuppressant it is likely to have a beneficial effect on unwanted toxicity. Furthermore, in cases where the cells expressing the protein are being used to achieve immunosuppression, notably with Treg cells expressing a CAR or TCR, the immunosuppressive properties of rapamycin and its analogues may be beneficial.

As indicated above, sequences for FRB and FKBP domains are known in the art. For example, FKBP/FRB may have or may comprise a sequence as shown in any one of SEQ ID NO: 1 to SEQ ID NO: 5, or a variant thereof. SEQ ID NO: 1 is the native (wild-type) human FKBP12 domain; SEQ ID NO: 2 is the wild-type FRB segment of mTOR; SEQ ID NO: 3 is FRB with T to L substitution at 2098 which allows binding to AP21967; SEQ ID NO: 4 is a FRB segment of mTOR with T to H substitution at 2098 and to W at F at residue 2101 of the full mTOR which binds Rapamycin with reduced affinity to wild type; SEQ ID NO: 5 is a FRB segment of mTOR with K to P substitution at residue 2095 of the full mTOR which binds Rapamycin with reduced affinity.

Additional FRB and FKBP domain sequences include the following SEQ ID NOs and variants thereof:

SEQ ID NO: 6 represents a FRB sequence as shown as part of the protein of SEQ ID NO. 1 of WO 2016/135470;

SEQ ID NO: 7 represents a FKBP sequence which is contained in the protein of SEQ ID NO. 4 of WO 2016/135470;

SEQ ID NO: 8 a so-called “codon-wobbled” FKBP sequence, in which the codons encoding the FKBP domain have been altered to prevent recombination (see SEQ ID NO.4 of WO 2016/135470);

SEQ ID NO: 88 represents a FKBP sequence as shown as part of the protein of SEQ ID NO. 1 of WO 2016/135470;

SEQ ID NO: 89 represents a FKBP sequence as shown as part of the protein of SEQ ID NO. 2 of WO 2016/135470.

SEQ ID NOs: 101-103 represent alternative FRB sequences.

Variant sequences may have at least 80%, 85%, 90%, 95%, 98% or 99% sequence identity to SEQ ID NO: 1 to 8, 88, 89, or 101-103 provided that the sequences provide an effective dimerization system. That is, provided that the sequences facilitate sufficient co-localisation of the two chimeric proteins to allow dimerization of the two signalling domains. Particularly, FRB and/or FKBP variants may retain the ability to bind to rapamycin or an analogue thereof, e.g. have at least 30, 40, 50, 60, 70, 80 or 90% of the binding affinity of FRB or FKBP to rapamycin/rapamycin analogue.

The “wild-type” FRB domain shown as SEQ ID NO: 2 comprises amino acids 2025-2114 of human mTOR. Using the amino acid numbering system of human mTOR, the FRB sequence of the chimeric protein may comprise an amino acid substitution at one of more of the following positions: 2095, 2098 and 2101.

The variant FRB used in the chimeric protein of the invention may comprise one of the following amino acids at positions 2095, 2098 and 2101:
2095: K, P, T or A;
2098: T, L, H or F;
2101: W or F.

Bayle et al (as above) describe the following FRB variants, annotated according to the amino acids at positions 2095, 2098 and 2101 (see Table 1 of Bayle): KTW, PLF, KLW, PLW, TLW, ALW, PTF, ATF, TTF, KLF, PLF, TLF, ALF, KTF, KHF, KFF, KLF. These variants are capable of binding rapamycin and rapalogs to varying extents, as shown in Table 1 and FIG. 5A of Bayle et al. The chimeric protein herein may comprise any one of these FRB variants.

Whilst in many cases, rapamycin is an attractive CID, there may nonetheless be situations where it is desired or preferable to use another CID or other heterodimerization domains, for example in certain clinical situations. Thus, systems based on other CIDs and other heterodimerization domains are available and may be used.

For example, FK506 is an inhibitor of the protein calcineurin. Thus, calcineurin also binds to FK506 (when FK506 is bound by FKBP) and may also be used to provide, or as the basis for, a heterodimerization domain which binds to FK506 as CID, along with FKBP as the cognate heterodimerization domain. Thus, in another embodiment the CID is FK506, and Ht1 and Ht2 are protein domains which bind thereto. In such an embodiment, one of the heterodimerization domains (Ht1 or Ht2) may be or comprise calcineurin or a FK506-binding fragment thereof, and the other heterodimerization domain (Ht2 or Ht1) may be or comprise FKBP (which may be as defined above, and includes for example FKBP12). The term “calcineurin” include variants and derivatives thereof. Such variants may include amino acid sequences having one or more amino acid modifications (e.g. substitutions, additions and/or deletions) relative to the native sequence. In this context a variant or derivative of calcineurin retains the ability of the parent molecule to bind to FK506. Calcineurin A is the 59 kDa catalytic subunit of the calcineurin protein and the heterodimerization domain may be or may comprise calcineurin A or a FK506-binding fragment thereof. By way of representative example, the sequence of an isoform of human calcineurin A (UniprotKB-Q08209) is shown in SEQ ID NO: 90.

In another embodiment, the CID may be the calcineurin inhibitor cyclosporine, also known as cyclosporine A (CsA), and Ht1 and Ht2 may be protein domains which bind thereto. In this regard, cyclophilins (CyPs) are a family of proteins characterised by their ability to bind to bind to CsA. Calcineurin activity is inhibited by CsA when bound to CyP (in other words, calcineurin binds to CsA when it is bound to CyP). Thus, cyclophilins may be used to provide a heterodimerization domain capable of binding to CsA as CID. In such an embodiment, one of the heterodimerization domains (Ht1 or Ht2) may be or comprise calcineurin or a CsA-binding fragment thereof (more particularly a CsA-CyP binding fragment), and the other heterodimerization domain (Ht2 or Ht1) may be or comprise cyclophilin or a CsA-binding fragment thereof. As noted above the calcineurin may be a variant or derivative of a native, or wild-type calcineurin molecule. In this context a variant or derivative of calcineurin retains the ability of the parent molecule to bind to CsA. The heterodimerization domain may be or may comprise calcineurin A or a CsA- (or CsA-CyP)-binding fragment thereof. Various isoforms of cyclophilin exist and the term “cyclophylin” (CyP) includes all such isoforms, e.g. cyclophilin A, B, C and D, and variants and derivatives thereof. In particular the cyclophilin may be CyPA. Variants of CyP include amino acid sequences having one or more amino acid modifications (e.g. substitutions, additions and/or deletions) relative to the native sequence. In this context a variant or derivative of cyclophilin retains the ability of the parent molecule to bind to CsA. For use in CID systems, a cyclophilin heterodimerization domain may be provided as a fusion protein with Fas (CyP-Fas). A representative sequence of cyclophilin, isoform 1 (UniprotKB P62937-1) is shown in SEQ ID NO: 91.

Based on the CsA and FK506 systems discussed above, a “heterodimeric CID” molecule has been developed which combines a protein-binding interface from FK506 with a protein-binding interface from CsA, to form the molecule FKCsA (Belshaw, et al., 1996, PNAS, 93, 4604-4607). Accordingly, in a further embodiment, the CID is FKCsA, and Ht1 and Ht2 are protein domains which bind thereto. In such an embodiment, one of the heterodimerization domains (Ht1 or Ht2) may be or comprise cyclophilin or a CsA-binding fragment thereof, and the other heterodimerization domain (Ht2 or Ht1) may be or comprise FKBP (Cyclophilin and FKBP and their respective fragments and variants may be as defined above, and include FKBP12 and CyP-Fas).

Further CID dimerization systems which may be used include those based on the self-labelling proteins SNAP-tag, a mutant of the DNA Repair protein O6-alkylguanine-DNA-alkyltransferase (available from New England Biolabs; and see Gautier et al., 2008, Chem. Biol. 15, 128-136), and HALO-Tag, a haloalkane dehalogenase derivative designed to covalently bind to synthetic ligands (available from Promega; and see Los, et al., 2008. ACS Chem. Biol. 3, 373-382). CIDs for SNAP-tag and HALO-tag have been developed, collectively termed HaXS, which function as chemical cross-linkers to bind SNAP-tag and HALO-tag fusion proteins together. Thus, in another embodiment the CID is a HaXS molecule, e.g. HaXS8, and Ht1 and Ht2 are protein domains which bind thereto. In such an embodiment, one of the heterodimerization domains (Ht1 or Ht2) may be or comprise a SNAP-tag and the other heterodimerization domain (Ht2 or Ht1) may be or comprise a HALO-tag.

A still further heterodimerization system which may be used is based on fusicoccin (FC) as CID, which binds simultaneously to a 14-3-3 protein and the C-terminal 52 amino acids (CT52) of a plant plasma membrane H+-ATPase (PMA) to stabilise the interaction between these two proteins. Accordingly, in another embodiment, the CID is fusicoccin, and Ht1 and Ht2 are protein domains which bind thereto. In such an embodiment, one of the heterodimerization domains (Ht1 or Ht2) may be or comprise a 14-3-3 protein or a FC-binding fragment thereof, and the other heterodimerization domain (Ht2 or Ht1) may be or comprise a C-terminal peptide of PMA, e.g. a CT52 peptide. The 14-3-3 protein may for example be tobacco 14-3-3 (T14-3-3c) or a truncation thereof (T14-3-3cAC, residues 1-242). The C-terminal peptide of PMA may be the CT52 fragment of tobacco H+ ATPase PMA2. The terms “14-3-3 protein”, C-terminal peptide of PMA″ and “CT52” include variants and derivatives thereof. Such variants may include amino acid sequences having one or more amino acid modifications (e.g. substitutions, additions and/or deletions) relative to the native sequence. In this context a variant or derivative retains the ability of the parent molecule to bind to fusicoccin. One such variant of CT52 of tobacco H+ ATPase PMA2 comprises three amino acid substitutions S939A, T955D and V9561. The tobacco 14-3-3 and CT52 interaction partners been described in the literature (Ottman et al., 2007, Mol. Cell 25(3), 427-440, and Truong et al., 2002, Proteins 49(3), 321-325). The full T14-3-3-3c sequence (UniProtKB- Q5KTN5) is shown in SEQ ID NO: 92. The CT52 sequence of Tobacco H+ATPase PMA2 as derived from UniProtKB-Q40409 is shown in SEQ ID NO: 93.

Variants of any amino acid sequence presented herein may have at least 80%, 85%, 90%, 95%, 98% or 99% sequence identity to the reference sequence (i.e. to a reference SEQ ID NO. as specified herein), for example to any one of SEQ ID NO: 90, 91, 92 or 93 as referred to above. In particular, such a variant retains the desired or required property of the parent molecule from which it is derived, i.e. the reference sequence. Thus, the variant sequence may have the stated % sequence identity provided that the variant sequence provides an effective dimerization system.

That is, provided that the sequences facilitate sufficient co-localisation of the two chimeric proteins to allow dimerization of the two signalling domains. Particularly, the variants may retain the ability to bind to their respective CID, e.g. have at least 30, 40, 50, 60, 70, 80 or 90% of the binding affinity of the parent molecule to calcineurin, cyclophilin or fusicoccin.

It will thus be seen that the chemical inducer of dimerization (CID) may be any molecule which induces heterodimerization between Ht1 and Ht2 on separate chimeric molecules having the same Ht1 and Ht2 domains. It may be a molecule to which both heterodimerization domains Ht1 and Ht2 may bind in any way. This may be for example affinity-based binding (in other words the CID may be a ligand), or the CID may be a molecule which allows interaction between the heterodimerization domains by other means, such as chemical cross-linking. Thus the binding may include covalent bonding, and the CID may be a chemical cross-linker (i.e. a bifunctional molecule which comprises a first group reactive with Ht1 and a second group reactive with Ht2).

The CID may be rapamycin or a rapamycin analogue (“rapalog”) which has improved or differing pharmadynamic or pharmacokinetic properties to rapamycin but have the same broad mechanism of action. The CID may be an altered rapamycin with engineered specificity for complementary FKBP12 or FRB—for example as shown in FIG. 2. Bayle et al (2006, as above) describes various rapalogs functionalised at C16 and/or C20.

Examples of such rapalogs in the first category include Sirolimus, Everolimus, Temsirolimus and Deforolimus. Examples of rapalogs in the second category include C-20-methyllyrlrapamycin (MaRap); 016(S)-Butylsulfonamidorapamycin (C16-BS-Rap); C16-(S)-3-mehylindolerapamycin (C16-iRap); and C16-(S)-7-methylindolerapamycin (AP21976/C16-AiRap).

Rapamycin is a potent immunosuppressive agent. Analogues of rapamycin are in every day clinical use. Modern rapalogs have excellent bioavailability and volumes of distribution. Although they are potent immunosuppressive agents, a short dose (to activate the signalling protein) would be expected to have minimal side-effects when treating conditions requiring an increased immune response, such as cancer (although long term dosing of rapamycin may not be desirable in such subjects/patients). As discussed above, the immunosuppressive effects would be beneficial when treating an inflammatory condition or disease, e.g. an autoimmune disease or transplant rejection, e.g. using a cell of the invention.

Other examples of CIDs include FK506, CsA, FKCsA and Fusicoccin, as discussed above and depicted in FIG. 9A-D. HaSX CIDs may be represented by HaSX8, also depicted in FIG. 9 E.

The present chimeric protein comprises a signalling domain, also referred to as an endodomain, which comprises a STAT5 association motif, a JAK1- and/or a JAK2-binding motif, and optionally a JAK3-binding motif.

As noted above, STAT5 is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the JAK1/JAK2 and JAK3 kinases via specific signalling domains present in the IL-2Rβ and IL-2Rγ chain, respectively. Although JAK1 (or JAK2) can phosphorylate STAT5 without the need of Jak3, STATS activity is increased by the transphosphorylation of both JAK1/JAK2 and JAK3, which stabilizes their activity.

“STAT5 association motif” as used herein refers to an amino acid motif which comprises a tyrosine and is capable of binding a STAT5 polypeptide. Any method known in the art for determining protein:protein interactions may be used to determine whether an association motif is capable of binding to STATS. For example, co-immunoprecipitation followed by western blot.

Suitably, the signalling domain may comprise two (e.g. at least two) or more STAT5 association motifs as defined herein. For example, the signalling domain may comprise two, three, four, five or more STAT5 association motifs as defined herein. In an embodiment, the signalling domain may comprise two or three STAT5 association motifs as defined herein.

Suitably, the STAT5 association motif may exist endogenously in a cytoplasmic domain of a transmembrane protein which may be used to provide the signalling domain of the chimeric protein herein. For example, the STAT5 association motif may be from an interleukin receptor (IL) receptor endodomain or a hormone receptor.

The signalling domain may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP_000869.1, SEQ ID NO: 9), amino acid numbers 265 to 459 of IL-7R α chain (NCBI REFSEQ: NP_002176.2, SEQ ID NO: 10), amino acid numbers 292 to 521 of IL-9R chain (NCBI REFSEQ: NP_002177.2, SEQ ID NO: 11), amino acid numbers 257 to 825 of IL-4R α chain (NCBI REFSEQ: NPJD00409.1, SEQ ID NO: 12), amino acid numbers 461 to 897 of IL-3R β chain (NCBI REFSEQ: NP_000386.1, SEQ ID NO: 13) and/or amino acid numbers 314 to 502 of IL-17R β chain (NCBI REFSEQ: NP_061195.2, SEQ ID NO: 14) may be used. It will be appreciated by a skilled person that any one or more of these sequences can be used. The entire region of the cytoplasmic domain of an interleukin receptor chain may be used.

SEQ ID NO: 15 represents an IL7RA 2Y truncated sequence, which may also be used.

The signalling domain may comprise one or more STAT5 association motifs that comprise an amino acid sequence shown as SEQ ID NO: 9-15 or a variant which is at least 80, 85, 90, 95, 96, 97, 98 or 99% identical to SEQ ID NO: 9-15. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 9-15. The variant or derivative may be capable of binding STAT5 to a similar or the same level as one of SEQ ID NO: 9-15 or may be capable of binding STAT5 to a greater level than an amino acid sequence shown as one of SEQ ID NO: 9-15 (e.g. increased by at least 10%, at least 20%, at least 30%, at least 40% or at least 50%).

For example, the STAT5 association motif may be from any one or more of IL2Rβ, IL7Rα, IL-3Rβ. (CSF2RB), IL-9R, IL-17Rβ, erythropoietin receptor, thrombopoietin receptor, growth hormone receptor and prolactin receptor. A chimeric protein may, for example, comprise STAT association motifs from both IL2Rβ and IL7Rα.

The STAT5 association motif may comprise the amino acid motif YXXF/L (SEQ ID NO: 16); wherein X is any amino acid.

Suitably, the STAT5 association motif may comprise the amino acid motif YCTF (SEQ ID NO: 17), YFFF (SEQ ID NO: 18), YLSL (SEQ ID NO: 19), or YLSLQ (SEQ ID NO: 20).

The signalling domain may comprise one or more STAT5 association motif comprising the amino acid motif YCTF (SEQ ID NO: 17), YFFF (SEQ ID NO: 18), YLSL (SEQ ID NO: 19), and/or YLSLQ (SEQ ID NO: 20).

The signalling domain may comprise a first STAT5 association motif comprising the amino acid motif YLSLQ (SEQ ID NO: 20) and a second STAT5 association motif comprising the amino acid motif YCTF (SEQ ID NO: 17) or YFFF (SEQ ID NO: 18).

The signalling domain may comprise the following STAT5 association motifs: YLSLQ (SEQ ID NO: 20), YCTF (SEQ ID NO: 17) and YFFF (SEQ ID NO: 18).

“JAK1- and/or a JAK2-binding motif” as used herein refers to a BOX motif which allows for tyrosine kinase JAK1 and/or JAK2 association. Suitable JAK1- and JAK2-binding motifs are described, for example, by Ferrao & Lupardus (Frontiers in Endocrinology; 2017; 8(71); which is incorporated herein by reference).

As noted above, the JAK1 and/or JAK2-binding motif may occur endogenously in a cytoplasmic domain of a transmembrane protein.

For example, the JAK1 and/or JAK2-binding motif may be from Interferon lambda receptor 1 (IFNLR1), Interferon alpha receptor 1 (IFNAR), Interferon gamma receptor 1 (IFNGR1), IL10RA, IL20RA, IL22RA, Interferon gamma receptor 2 (IFNGR2) or IL10RB.

The JAK1-binding motif may comprise or consist of an amino acid motif shown as SEQ ID NO: 21-27 or a variant thereof which is capable of binding JAK1.

(SEQ ID NO: 21)
KVLKCNTPDPSKFFSQLSSEHGGDVQKWLSSPFPSSSFSPGGLAPEISP
LEVLERDK
(SEQ ID NO: 22)
NPWFQRAKMPRALDFSGHTHPVATFQPSRPESVNDLFLCPQKELT
(SEQ ID NO: 23)
GYICLRNSLPKVLNFHNFLAWPFPNLPPLEAMDMVEVIYINR
(SEQ ID NO: 24)
PLKEKSIILPKSLISVVRSATLETKPESKYVSLITSYQPFSL
(SEQ ID NO: 25)
RRRKKLPSVLLFKKPSPFIFISQRPSPETQDTIHPLDEEAFLK
(SEQ ID NO: 26)
YIHVGKEKHPANLILIYGNEFDKRFFVPAEKIVINFITLNISDDS
(SEQ ID NO: 27)
RYVTKPPAPPNSLNVQRVLTFQPLRFIQEHVLIPVFDLSGP

The variant of SEQ ID NO: 21-27 may comprise one, two or three amino acid differences compared to any of SEQ ID NO: 21-27 and retain the ability to bind JAK1.

The variant may be at least 80, 85, 90, 95, 96, 97, 98 or 99% identical to any one of SEQ ID NO: 21-27 and retain the ability to bind JAK1.

In a preferred embodiment, the JAK1-binding domain comprises or consists of SEQ ID NO: 21 or a variant thereof which is capable of binding JAK1.

For example, the variant may be capable of binding JAK1 to at least 10%, at least 20%, at least 30%, at least 40%, at least 50%, at least 60%, at least 70%, at least 80%, or at least 90% of the level of a corresponding, reference sequence. The variant or derivative may be capable of binding JAK1 to a similar or the same level as a corresponding, reference sequence or may be capable of binding JAK1 to a greater level than a corresponding, reference sequence (e.g. increased by at least 10%, at least 20%, at least 30%, at least 40% or at least 50%).

The JAK2-binding motif may comprise or consist of an amino acid motif shown as SEQ ID NO: 28-30 or a variant therefore which is capable of binding JAK2.

(SEQ ID NO: 28)
NYVFFPSLKPSSSIDEYFSEQPLKNLLLSTSEEQIEKCFIIEN
(SEQ ID NO: 29)
YWFHTPPSIPLQIEEYLKDPTQPILEALDKDSSPKDDVWDSVSIISFPE
(SEQ ID NO: 30)
YAFSPRNSLPQHLKEFLGHPHHNTLLFFSFPLSDENDVFDKLSVIAEDS
ES

The variant of SEQ ID NO: 28-30 may comprise one, two or three amino acid differences compared to any of SEQ ID NO: 28-30 and retain the ability to bind JAK2.

For example, the variant may be capable of binding JAK2 to at least 10%, at least 20%, at least 30%, at least 40%, at least 50%, at least 60%, at least 70%, at least 80%, or at least 90% of the level of a corresponding, reference sequence. The variant or derivative may be capable of binding JAK2 to a similar or the same level as a corresponding, reference sequence or may be capable of binding JAK2 to a greater level than a corresponding, reference sequence (e.g. increased by at least 10%, at least 20%, at least 30%, at least 40% or at least 50%).

Any method known in the art for determining protein:protein interactions may be used to determine whether a JAK1- or JAK2-binding motif is capable of binding to a JAK1 or JAK2. For example, co-immunoprecipitation followed by western blot

Suitably, the signalling domain may comprise an IL2Rβ endodomain shown as SEQ ID NO: 1; or a variant which has at least 80% sequence identity to SEQ ID NO: 1.

The variant may be at least 80, 85, 90, 95, 96, 97, 98 or 99% identical to SEQ ID NO: 1.

Suitably, the signalling domain may comprise a truncated IL2Rβ endodomain shown as any one of SEQ ID NO: 31 or 32 or a variant of any one of SEQ ID NO: 31 or 32 which has at least 80% sequence identity thereto. SEQ ID NO: 31 represents a IL2RB truncated variant with a Y510 mutation. SEQ ID NO: 32 represents a IL2RB truncated variant with Y510 and Y392 mutations.

The variant may be at least 80, 85, 90, 95, 96, 97, 98 or 99% identical to SEQ ID NO: 31 or 32.

STAT5 activity is increased by the transphosphorylation of both a JAK1/2 and JAK3, as this stabilizes their activity. As noted above, the signalling domain may further comprise a JAK3-binding motif. “JAK3-binding motif” as used herein refers to a BOX motif which allows for tyrosine kinase JAK3. Suitable JAK3-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK3. For example, co-immunoprecipitation followed by western blot.

The JAK3-binding motif may occur endogenously in a cytoplasmic domain of a transmembrane protein.

For example, the JAK3-binding motif may be from an IL-2Rγ polypeptide. A functional truncated or variant IL2Rγ polypeptide may be used within the signalling domain of the chimeric protein, wherein the functional truncated or variant IL2Ry polypeptide retains JAK3-binding activity (e.g. at least 20, 30, 40, 50, 60, 70, 80, 90 or 95% of binding activity of IL2Rγ). Particularly, a truncated IL2Rγ comprising a JAK3-binding motif and a truncated IL2Rβ comprising a STAT5 association motif, and a JAK1- and/or JAK2-binding motif may be comprised in the signalling domain of a chimeric protein as defined herein. Functional truncations may provide an advantage of reducing construct size for expression.

The JAK3-binding motif may comprise or consist of an amino acid motif sequence shown as SEQ ID NO: 33 or SEQ ID NO: 34 ora variant thereof which is capable of binding JAK3 (e.g. a functional variant or fragment having at least 80, 85, 90, 95 or 99% identity to SEQ ID NOs: 33 or 34).

The variant may be at least 80, 85, 90, 95, 96, 97, 98 or 99% identical to SEQ ID NO: 33 or SEQ ID NO: 34.

In a particular embodiment, the signalling domain comprises one or more JAK1-binding domains and at least one JAK3-binding domain/motif (e.g. at least 2 or 3 JAK3-binding domains/motifs).

It will be appreciated by a skilled person that the polynucleotide sequence encoding the JAK3-binding domain may be positioned upstream or downstream (5′ or 3′) of the polynucleotide sequence encoding the STAT 5 association motif and JAK1 and/or JAK2 binding motifs. Typically, the JAK1 and/or 2 motifs would be upstream (5′) of STATS, but this may be varied in certain constructs. Particularly, the polynucleotide encoding the JAK3-binding domain may be positioned downstream (3′) of the polynucleotide encoding the STAT 5 association motif and the JAK1 and/or JAK2-binding motifs. Thus, alternatively viewed, in the chimeric protein as described herein, the JAK3-binding domain may be N or C terminal to the STAT5 association motif and the JAK1 and/or JAK2-binding domains, preferably C terminal. In one embodiment, the JAK3-binding domain and the STAT5 association motif/JAK1 and/or JAK2-binding domains are positioned directly adjacent to one another (i.e. are not separated distally by sequence). In a particular embodiment, the JAK3 binding domain is translated in reverse orientation, thus the JAK3 binding motif may comprise a sequence in the reverse orientation to SEQ ID NOs: 33 or 34 (e.g. as shown in SEQ ID NO: 87). The polynucleotide encoding the signalling domain may thus comprise nucleotide sequences in the following order: 5′-3′ JAK1, 5′-3′ STATS association motif, 3′-5′ JAK3.

Further, a skilled person will appreciate that the JAK3-binding domain and the STAT5 association motif and JAK-1 and/or JAK2-binding domains, may be positioned at any location within the cytoplasmic domain of the chimeric protein, e.g. proximal to the membrane, or separated from the membrane by additional sequence, e.g. by the binding domain or a part thereof. In one embodiment, it is possible for the domains to extend into the transmembrane region.

Thus, various configurations of the Ht1, Ht2, and signalling domains of the chimeric protein are possible. This may depend upon whether or not the binding domain is a 1-part or 2-part domain.

In representative embodiments the signalling domain may comprise, from N- to C-terminal:
(i) JAK1 and/or JAK2 motif-STAT5 motif;
(ii) JAK1 and/or JAK2 motif-STAT5 motif-JAK3 motif, particularly in reverse orientation;
(iii) JAK1 motif-STAT5 motif; or
(iv) JAK1 motif-STAT5 motif-JAK3 motif, particularly in reverse orientation;
   wherein the motifs are linked directly or indirectly to one another. Where the motifs are linked indirectly, they may be linked by a linker.

In a particular embodiment, a linker or a hinge may be present between the JAK3-binding motif and the STAT5 association motif/JAK1 and/or JAK2 binding motifs. The linker or hinge may comprise or consist of at least 2, 3, 4, 5, 6, 7, 8, 9, 10, 11, 12, 13, 14, 15, 16, 17, 18, 19, 20, 25 or 30 amino acids, e.g. at least 2, 3, 4, 5, 6, 7, 8, 9 or 10 glycine residues. In a most particular embodiment, the signalling domain comprises a first amino acid sequence derived from IL2Rγ comprising a JAK3-binding domain (e.g. SEQ ID NOs: 33 or 34) and a second amino acid sequence derived from IL2Rβ comprising a STAT5 association motif and a JAK1 and/or JAK2-binding motif (e.g. SEQ ID NOs: 31or 32), where the first and second amino acid sequences are connected or joined by a linker or hinge. For example, the signalling domain may comprise SEQ ID NO: 33 and SEQ ID NO: 31 wherein SEQ ID NOs: 33 and 31 are connected by a linker or hinge.

As indicated above, the binding domain or parts thereof and the signalling domain may be joined to each other via a linker. Where Ht1 and Ht2 are present together in a single 1-part domain, these may also be joined together directly, or by a linker. Further, if the chimeric protein comprises other domains or sequences, for example a transmembrane (TM) domain or extracellular domain (Exo domain), then such domains or sequences may be linked directly or indirectly via a linker. Linker sequences are discussed in more detail below.

Various configurations of the chimeric protein are possible. For example, both the signalling domain and the binding domain may be intracellular. The binding domain may be proximal to the membrane (as shown in FIG. 1A for example) or distal to the membrane (as shown in FIG. 1B for example). Alternatively, as mentioned above, the signalling domain may extend into the TM domain of the protein. The signalling domain is depicted as comprising, N- to C-terminal JAK1-STAT5, but this may be modified to introduce a JAK3 domain linked at the C-terminus of STAT5, particularly in reverse orientation. In a further example, the binding domain may be extracellular, as depicted for example in FIG. 1C. FIG. 1D depicts a configuration of a membrane protein with a 2-part binding domain, wherein both binding and signalling domains are intracellular and where one of Ht1 and Ht2 is at N-terminus of the signalling domain, and the other is at the C-terminus.

It will be understood that the configurations depicted in FIG. 1 are exemplary only, and may be modified or altered. For example, Ht1 and Ht2 are shown in FIG. 1 as FKBP and FRB respectively, but this may equally be vice versa.

In some embodiments, Ht1 and/or Ht2 are contained in a hinge domain of the chimeric protein.

In representative embodiments, the chimeric protein may have the following configurations:
(i) where the chimeric protein comprises (a) a TM domain or (b) a TM domain and an Exo domain, the JAK1 and/or JAK2 motifs in the signalling domain may be linked N-terminal to the STAT5 motif;
(ii) where the signalling domain of the chimeric protein comprises a JAK3 motif, this is linked to the other end of the STAT5 motif than the JAK1 and/or JAK2 motif.

The skilled person would be able to design an appropriate configuration for the chimeric protein, depending on the nature of the Ht1 and Ht2 domains and the signalling domain, for example whether or not the signalling domain includes a JAK3 binding motif. Generally speaking, for 2-sided dimerization, where the binding domain is in 2 parts and Ht1 and Ht2 are separated, the signalling domain includes a JAK3 binding motif.

The purpose of the dimerization of the binding domain is to bring together 2 monomers of the signalling domain, and to allow them to dimerize to form a functional signalling protein. The dimerization between the signalling domains does not require a physical association between the two signalling domain monomers, in the sense of the 2 domains becoming physically linked or joined to one another—it suffices that they are brought into proximity together in such a manner that they can act together to mediate a signal in the cell. The dimerization between the two signalling domain monomers thus includes a functional association between them.

The skilled person is well aware of the requirements for dimerization of the signalling domains, or more particularly the individual motifs thereof. Particular motifs may homo-dimerize with one another, but this is not necessarily the case. In the case of a 1-part binding domain, where there is 1-sided dimerization, the signalling domain may be designed such that individual motifs homo-dimerize. Thus, as depicted in FIGS. 1A, B, C and D for example, STAT5 may homodimerize, as may the JAK1 and/or 2 motifs. However, the motifs do not need to homodimerize with one another, and whilst typically in a chimeric protein, STAT5 motifs will be designed to homo-dimerize with one another, STAT5 may for example heterodimerize with JAK1 or JAK2. Further, JAK1 or JAK2 may heterodimerize with JAK3. JAK1 dimerization with JAK3 is depicted in FIG. 1D for example). To achieve functional dimerization of JAK1 and JAK3, from separate chimeric protein chains, JAK3 is typically translated in reverse orientation.

The signalling domain may provide other signalling functions (e.g. those capable of providing a pro-survival or persistence signal, a signal which maintains cell phenotype or induces activation or function in addition to providing a STAT5 signal), and thus may comprise further domains which are capable of providing such signalling functions. The signalling domain however may not comprise any suicide or safety switch moiety or domain, and thus particularly does not comprise any killing or suicide function.

The signalling domain for example, may additionally comprise an intracellular signalling domain such as ζ chain endodomain of the T-cell receptor or any of its homologs (e.g., η chain, FcϵR1γ and β chains, MB1 (Igα) chain, B29 (Igβ) chain, etc.), CD3 polypeptide domains (Δ, δ and ϵ), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcyRlll, FcsRl, cytoplasmic tails of Fc receptors, immunoreceptor tyrosine-based activation motif (ITAM) bearing cytoplasmic receptors or combinations thereof.

Preferably, the additional intracellular signaling domain comprises the intracellular signaling domain of a human CD3 zeta chain, which in one embodiment comprises or consists of the following sequence:

UNIPROT: P20963, CD3Z_HUMAN, position 31-143

(SEQ ID NO: 81)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

In one embodiment, the intracellular signaling domain comprises at least 85, 90, 95, 97, 98 or 99% identity to SEQ ID NO: 81.

The intracellular signaling domain of the CAR may comprise the following CD28 signaling domain:

(SEQ ID NO: 82)
RSKRSRLLHSDYMNMTPRRPGPTRKHYQPYAPPRDFAAYRS

In one embodiment, the intracellular signaling domain comprises a signaling motif which has at least 85, 90, 95, 97, 98 or 99% identity to SEQ ID NO: 82.

The intracellular signaling domain of the CAR may comprise the following CD27 signaling domain:

(SEQ ID NO: 83)
QRRKYRSNKGESPVEPAEPCHYSCPREEEGSTIPIQEDYRKPEPACSP.

In one embodiment, the intracellular signaling domain comprises a signaling motif which has at least 85, 90, 95, 97, 98 or 99% identity to SEQ ID NO: 83.

Additional intracellular signaling domains will be apparent to those of skill in the art and may be used in connection with alternate embodiments of the invention.

A skilled person will appreciate that although the signalling domain as described herein provides a STAT5 signal to a cell in which the chimeric protein is expressed (upon provision of the CID), it would also be possible to provide other pro-survival, or phenotype maintenance signals to a cell using the inducible system described herein, in place of (or as well as the STAT5 signal). Thus, it would be possible for example to provide a STAT3 signal to a cell (particularly a Tcon cell), using a system of the invention, where the signalling domain may comprise YXXQ (SEQ ID NO: 84), where X is any amino acid, for example YRHQ (SEQ ID NO: 85). Alternatively, STAT1 or STAT4 signalling may be provided in a similar manner by a signalling domain of the invention.

In this aspect, the signalling domain may provide transcription factor activity to the cell in which it is expressed, e.g. a transcription factor which has importance for phenotype or function of the cell. For Tregs for example, the signalling domain may additionally be capable of providing the cell with FOXP3, c-Rel, Runx, Ets-1, CREB, NFAT and/or JunB (directly or indirectly). Particularly, the signalling domain may be capable of providing a FOXP3 activating or inducing signal to the cell. In one aspect, the signalling domain may comprise FOXP3 (or any functional variant, truncation or isoform thereof), wherein the FOXP3 may be cleavable from the chimeric protein upon induction with a CID (for example, using a Notch system). In this instance, the signalling domain may be present at the C terminus of the chimeric protein, and any cleavable portion (e.g. FOXP3) would be present at the C-terminus of the signalling domain.

The chimeric protein advantageously comprises at least a part of a transmembrane domain, and this may further if desired be combined with an extracellular domain. In the case of the latter, the chimeric protein may also comprise a hinge domain to hold the extracellular domain away from the cell surface. Generally speaking, the transmembrane domain is a domain which spans the phospholipid bilayer of the cell membrane. It may thus be regarded as the part of a protein chain of a transmembrane protein which lies within a cell surface membrane. The hinge and transmembrane domains may comprise the hinge and transmembrane sequences from any protein which has a hinge domain and/or a transmembrane domain, including any of the type I, type II or type III transmembrane proteins.

The transmembrane domain of the chimeric protein may also comprise an artificial hydrophobic sequence. Additional transmembrane domains will be apparent to those of skill in the art. The TM domain may for example be selected from any of those typically used in CARs. Examples of transmembrane (TM) regions which may be used are: 1) The CD28 TM region (Pule et al, Mol Ther, 2005, November; 12(5):933-41; Brentjens et al, CCR, 2007, Sep. 15; 13(18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4). The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; !mai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64.). Other transmembrane domains which may be used include those from CD4, CD45, CD9, CD16, CD22, CD33, CD64, CD80, CD86, or CD154. The transmembrane domain may further be derived from IL2RB.

A hinge domain may conveniently be obtained from the same protein as the transmembrane domain, although this is not essential.

By way of example the transmembrane domain may be derived from the CD28 transmembrane domain, and may comprise the amino acid sequence shown as SEQ ID NO: 35 or a variant which is at least 80% identical to SEQ ID NO: 35 The variant may be at least 80. 85, 90, 95, 97, 98 or 99% identical to SEQ ID NO: 35.

Alternatively, the chimeric protein may comprise a domain derived from the CD8α transmembrane domain. Thus, the transmembrane domain may comprise the amino acid sequence shown as SEQ ID NO: 36 which represents amino acids 183 to 203 of human CD8α, or a variant which is at least 80% identical to SEQ ID NO: 36. Suitably, the variant may be at least 85, 90, 95, 97, 98 or 99% identical to SEQ ID NO: 36.

The CD8α transmembrane domain may be combined with a CD8α hinge domain. In an embodiment the chimeric protein comprises a combined CD8α hinge and transmembrane domain sequence as shown in SEQ ID NO: 37, or a variant thereof which has at least 80% sequence identity thereto. The variant may be at least 85, 90, 95, 97, 98 or 99% identical to SEQ ID NO: 37. SEQ ID NO: 37 comprises a modified hinge domain comprising 2 amino acid substitutions of cysteine residues relative to the wild-type CD8α hinge sequence. The modified CD8α hinge domain sequence is shown in SEQ ID NO: 38. The wildtype CD8α hinge domain sequence is shown in SEQ ID NO: 39. A variant of such hinge sequences having at least 80% sequence identity to SEQ ID NO: 38 or 39 may be used.

An example of a CD28 hinge and transmembrane sequence which may be used is SEQ ID NO: 40 or a variant thereof which is at least 80% identical to SEQ ID NO: 40. The variant may be at least 85, 90, 95, 97, 98 or 99% identical to SEQ ID NO: 40.

By way of further example, the chimeric protein may comprise a native or modified CD8α hinge domain and a CD28 transmembrane domain, or a CD28 hinge domain and CD8α transmembrane domain, for example based on the sequences given above.

Other hinge domains which may be used include those from CD4, CD7, or an immunoglobulin, or a part or variant thereof.

More broadly, as indicated above the chimeric protein may comprise a membrane-targeting moiety. Such a moiety may be for example an amphipathic α-helix, a hydrophobic loop, or a lipid anchor, e.g. a myristate, It may be any moiety which is capable of associating with, e.g. binding to, or inserting into the cell membrane, or in any way associating with any component of a cell membrane.

The extracellular (“Exo”) domain may be any desired extracellular domain. It may be a functional domain, or it may be non-functional. That is, the domain may be included simply to provide as structural element to the protein, without necessarily imparting any functional property or activity to it.

In certain embodiments of any of the chimeric proteins described herein the extracellular domain is not an antigen binding domain, or it is not a binding domain derived from a receptor. Accordingly, in some embodiments the extracellular domain is not a TCR, nor a binding domain as would or could be used in a CAR. Thus, in some embodiments the extracellular domain is not a binding domain derived from an antibody. In a particular embodiment the extracellular domain is not a ScFv. Thus, it can be seen that in such embodiments the chimeric protein herein is not a CAR. Further, in such embodiments the chimeric protein is not a TCR-derived chimeric receptor (i.e. not a TCR-CAR construct).

Whilst in some embodiments of the protein, the binding domain comprising Ht1 and Ht2 may be extracellular, it will be understood that is distinct from an Exo domain which may be present in the protein. Thus, the protein may comprise a separate extracellular domain to the binding domain. The Exo domain as referred to herein is separate from the binding domain.

The extracellular domain of the chimeric protein may comprise a safety switch polypeptide. A safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the chimeric protein (and optionally a CAR or other receptor which is co-expressed by the cell along with the present chimeric protein), which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9 as is known in the art. However, in other examples the suicide moiety may be, or may comprise an epitope which is recognised by a cell-deleting antibody or other binding molecule capable of eliciting deletion of the cell.

The term “delete” as used herein in the context of cell deletion is synonymous with “remove” or “ablate” or “eliminate” The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. More particularly, the polypeptide may comprise two CD20 epitopes R1 and R2 that are spaced apart by a linker L.

Safety switches based on Rituximab epitopes are described in WO2013/15339. Peptides which mimic the epitope recognised by Rituximab (so-called mimotopes) have been developed, and these were used in WO2013/15339 as a suicide moiety in a combined suicide-marker polypeptide construct also comprising a CD34 minimal epitope as a marker moiety. Specifically, WO2013/15339 discloses a polypeptide termed RQR8, having the sequence set forth in SEQ ID NO: 41, which comprises two CD20 minimal epitopes, separated from one another by spacer sequences and an intervening CD34 marker sequence, and further linked to a stalk sequence which allows the polypeptide to project from the surface of a cell on which it is expressed. The safety switch polypeptide may be RQR8 or a variant thereof having at least 80% sequence identity thereto, e.g. at least 85, 88, 90, 95, 96, 97, 98, or 99% sequence identity thereto. Other safety switch polypeptides which may be used include those described in our co-pending UK patent application No. 2007842.4.

As noted above the various domains, and individual parts of the domains (e.g. the motifs in the signalling domain) may be linked to one another by linkers. Thus, the chimeric protein may contain one or more linkers. Typically, it will contain at least one linker. Notably the protein may contain a linker linking the binding domain, or a part thereof, to the signalling domain. For example, the chimeric protein may contain a linker linking a 1-part binding domain to the signalling domain, and a linker linking Ht1 to Ht2. In another example, the chimeric protein may comprise a 2-part binding domain wherein each of Ht1 and Ht2 are linked by a linker to the signalling domain. Further, the chimeric protein may comprise a linker linking the binding domain or signalling domain to TM domain (although in an embodiment a signalling domain may be linked directly to a TM domain). Still further, in another example, an extracellular binding domain may be linked to both an Exo domain and a TM domain, that is it may internally, between an Exo domain and a TM domain. In a further example, an extracellular binding domain may be linked at the end of an Exo domain, such that the binding domain forms the terminus of the protein, e.g. it may lie at the N-terminal end of the chimeric protein. In such a case, the binding domain may be linked by a linker to the Exo domain.

A linker as referred to herein is an amino acid sequence which links one domain or part of the protein to another. The linker sequence may be any amino acid sequence which functions to link, or connect, two domains or parts thereof together, such that they may perform their function. Thus a linker may space apart the elements which are linked, for example to allow them to bind to their target. For example, a linker may allow Ht1 and Ht2 from separate chimeric protein molecules to bind to a rapamycin molecule. Depending on the configuration of the protein, different linkers may be required, for example to connect the binding domain, or Ht1 and Ht2 individually to the signalling domain, or to one another, and/or to other domains (e.g. the Exo or TM domains),

The nature of the linker, in terms of its amino acid composition and/or sequence of amino acids may be varied and is not limited. However, the linker may be a flexible linker. It may thus comprise or consist of amino acids known to confer a flexible character to the linker (as opposed to a rigid linker).

Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27(10), 325-330; van Rosmalen et al., 2017, Biochemistry, 56,6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules. The most commonly used flexible linkers have sequences primarily composed of Ser and Gly residues (so-called “GS linkers”). However, many other flexible linkers have also been described (see Chen et a/, 2013, supra, for example), which may contain additional amino acids such as Thr and/or Ala, and/or Lys and/or Glu which may improve solubility. Any flexible linker known and reported in the art may be used.

The use of GS linkers, or more particularly GS (“Gly-Ser”) domains in linkers, may allow the length of the linker readily to be varied by varying the number of GS domain repeats, and so such linkers represent one suitable class of linkers. However, flexible linkers are not limited to those based on “GS” repeats, and other linkers comprising Ser and Gly residues dispersed throughout the linker sequence have been reported, including in Chen et al., supra.

In one embodiment, the linker sequence comprises at least one Gly-Ser domain composed solely of Ser and Gly residues. In such an embodiment, the linker may contain no more than 15 other amino acid residues, e.g. no more than 14, 13, 12, 11, 10, 9, 8, 6, 7, 5, or 4 other amino acid residues.

The Gly-Ser domain may have the formula:

(S)q-[(G)m-(S)m]n-(G)p
wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula:

(i) S-[(G)m-S]n;

(ii) [(G)m-S]n; or

(iii) [(G)m-S]n-(G)p
wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

S-[G-G-G-G-S]n
wherein n is an integer of at least one (preferably 1 to 8, or 1-6, 1-5, 1-4, or 1-3). In the formula above, the sequence GGGGS is SEQ ID NO: 46.

For example, S(GGGGS) 2 is SEQ ID NO: 42 and S(GGGGS) 4 is SEQ ID NO: 44.

However, it is not required for all linkers to be flexible, and in some cases the linker sequence is not a flexible linker sequence. Where the linker connects a binding domain, or Ht1 or Ht2 to a signalling domain, or to a TM or Exo domain it is preferably a flexible linker.

Although the length of the linker may not be critical, it may in some cases be desirable to have a shorter linker sequence, or a longer linker sequence, depending on what domains etc are being linked. For example, in some cases, for example where the linker connects Ht1 and Ht2, or where in a 2-part binding domain configuration the linker connects Ht1 and Ht2 individually to the signalling domain, or when linking the motifs within a signalling domain, the linker sequence may have a length of no more than 30, e.g. no more than 25, 24, 23, 22, 21 or 20 amino acids. Particularly, the linker connecting Ht1 and Ht2 may be less than 15, or 10 amino acids or alternatively viewed may be between 5-15 or 5-10 amino acids in length (e.g, for chimeric proteins having a one part binding domain, particularly for chimeric proteins having a one part binding domain C terminal to the signalling domain).

In other cases, for example where the linker connects a binding domain, or Ht1 or Ht2, to a signalling domain, or to a TM or Exo domain a longer linker sequence may be desired. This may particularly be the case where the binding domain is included internally, between two other domains, For example, a longer linker may be composed of, or may comprise, multiple repeats of a GS domain.

In some cases, the linker may be from any one of 2, 3, 4, 5 or 6 to any one of 24, 23, 22 or 21 amino acids in length. In other cases, it may be from any one of 2, 3, 4, 5 or 6 to any one of 21, 20, 19, 18, 17, 16, or 15 amino acids in length. In other cases, it may be intermediate between these ranges, from example from 6 to 21, 6 to 20, 7 to 20, 8-20, 9-20, 10-20, 8-18, 9-18, 10-18, 9-17, 10-17, 9-16, 10-16 etc. It may accordingly be within a range made up from any of the integers listed above.

In other cases, the linker may be of longer length, for example, from any one of 4, 5, 6, 7, 8, 9, 10, 12, 15, 20 to any one of 100, 90, 80, 70, 60, 50, 45, 40, 30, 28, 25 or 24 amino acids in length. In other cases, it may be intermediate between this range and any of the ranges indicated above. It may accordingly be within a range made up from any of the integers listed above.

As noted above, linkers may be designed or selected to allow the various domains and parts of the chimeric protein to function or to exert their effects, and so that the dimerizations may take place as set out above, namely that Ht1 from one protein molecule may heterodimerize to Ht2 from another protein molecule, and that the signalling domain monomers of the respective proteins may dimerize with one another. It is well within the routine skill of the person skilled in the art to design or select appropriate linkers, For example, a linker should provide sufficient flexibility so the signalling domains can dimerize, but not so much flexibility so that the energic barrier to such dimerization is not overcome.

The use of GS linkers, or more particularly GS (“Gly-Ser”) domains in linkers, may allow the length of the linker readily to be varied by varying the number of GS domain repeats, and so such linkers represent an advantageous type of linker to use. However, flexible linkers are not limited to those based on “GS” repeats, and other linkers comprising Ser and Gly residues dispersed throughout the linker sequence have been reported, including in Chen et al., supra.

A linker sequence may be composed solely of, or may consist of, one or more Gly-Ser domains as described or defined above. However, as noted above, the linker sequence may comprise one or more Gly-Ser domains, and additional amino acids. The additional amino acids may be at one or both ends of a Gly-Ser domain, or at one or both ends of a stretch of repeating Gly-Ser domains. Thus, the additional amino acid, which may be other amino acids, may lie at one or both ends of the linker sequence, e.g. they may flank the Gly-Ser domain(s). In other embodiments, the additional amino acids may lie between Gly-Ser domains. For example, two Gly-Ser domains may flank a stretch of other amino acids in the linker sequence. Further, as also noted above, in other linkers, GS domains need not be repeated, and G and/or S residues, or a short domain such as GS, may simply be distributed along the length or the sequence

Representative exemplary linker sequences are listed below:

(SEQ ID NO: 43)
ETSGGGGSRL
((SEQ ID NO: 45)
SGGGGSGGGGSGGGGS
S(GGGGS)₁₋₅ (where GGGGS is SEQ ID NO: 46)
(GGGGS)₁₋₅ (where GGGGS is SEQ ID NO: 46)
S(GGGS)₁₋₅ (where GGGS is SEQ ID NO: 47)
(GGGS)₁₋₅ (where GGGS is SEQ ID NO: 47)
S(GGGGGS)₁₋₅ (where GGGGGS is SEQ ID NO: 48)
(GGGGGS)₁₋₅ (where GGGGGS is SEQ ID NO: 48)
S(GGGGGGS)₁₋₅ (where GGGGGGS is SEQ ID NO: 49)
(GGGGGGS)₁₋₅ (where GGGGGGS is SEQ ID NO: 49)
(SEQ ID NO: 50)
G₆
(SEQ ID NO: 51)
G₈
(SEQ ID NO: 52)
KESGSVSSEQLAQFRSLD
(SEQ ID NO: 53)
EGKSSGSGSESKST
(SEQ ID NO: 54)
GSAGSAAGSGEF
(SEQ ID NO: 55)
SGGGGSAGSAAGSGEF
(SEQ ID NO: 56)
SGGGLLLLLLLLGGGS
(SEQ ID NO: 57)
SGGGAAAAAAAAGGGS
(SEQ ID NO: 58)
SGGGAAAAAAAAAAAAAAAAGGGS
(SEQ ID NO: 59)
SGALGGLALAGLLLAGLGLGAAGS
(SEQ ID NO: 60)
SLSLSPGGGGGPAR
(SEQ ID NO: 61)
SLSLSPGGGGGPARSLSLSPGGGGG
(SEQ ID NO: 62)
GSSGSS
(SEQ ID NO: 63)
GSSSSSS
(SEQ ID NO: 64)
GGSSSS
(SEQ ID NO: 65)
GSSSSS
(SEQ ID NO: 66)
SGGGGS.

For linking motifs within a signalling domain, the following linkers can be mentioned:

(SEQ ID NO: 46)
GGGGS
(SEQ ID NO: 67)
GGGGSGGGGSGGGGS
(SEQ ID NO: 68)
GGGGG
(SEQ ID NO: 69)
GGGGSGGGGS
(SEQ ID NO: 70)
GGGGSGGGGSGGGGSGGGGS
(SEQ ID NO: 71)
GGGGGGG
(SEQ ID NO: 72)
GGGGGGGGG

The chimeric protein may also comprise, or be expressed with, a signal peptide at the amino terminus (otherwise known as a leader sequence). A number of different signal sequences are known and reported in the art and it would be a matter of routine to select a signal peptide. The signal peptide may, for example, comprise or consist of the sequence shown as SEQ ID NO: 73

(MGTSLLCWMALCLLGADHAD).

Illustrative constructs are represented by SEQ ID NO: 99 or SEQ ID NO: 100 as used in the Examples herein. These constructs comprise an interaction domain comprising dimerization domains FRB and FKBP separated from each other by a 5 amino acid linker. The interaction domain is joined by a 17 amino acid linker to a transmembrane domain from IL2Rβ (IL2RB), which is linked to a signalling domain comprising a IL2RB truncated variant with a Y510 mutation (SEQ ID NO: 31). The depicted constructs also comprise an N-terminal modified safety switch sequence (p66 RSR) and are linked at the C-terminal to a P2A self-cleavage sequence and GFP for the purposes of expressing the constructs and detecting them in the experiments described. Representative constructs for use herein may be based on the constructs of SEQ ID NOs: 99 or 100, but omitting or substituting the ARSR, and/or omitting or substituting the GFP, for example for another protein of interest, as described in more detail below.

A second aspect herein provides a nucleic acid molecule comprising a nucleotide sequence which encodes the chimeric protein.

As used herein, the terms “polynucleotide” and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed. Nucleotide sequences encoding the various domains and motifs etc. described herein are known and available in the art, and any of these may be used or modified for use herein.

Nucleic acids according to the second aspect may comprise DNA or RNA. They may be single-stranded or double-stranded. They may also be polynucleotides which include within them synthetic or modified nucleotides. A number of different types of modification are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest. The terms “variant”, “homologue” or “derivative” in relation to a nucleotide sequence include any substitution of, variation of, modification of, replacement of, deletion of or addition of one (or more) nucleic acid from or to the sequence.

Nucleic acid molecules/polynucleotides/nucleotide sequences such as DNA nucleic acid molecules/polynucleotides/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

A nucleic acid construct may comprise the nucleic acid molecule together with one or more other nucleotide sequences, for example, regulatory sequences, e.g. expression control sequences, and/or other coding sequences. In particular, the other coding sequence may encode a protein of interest. This may be a therapeutic protein.

As noted above, a chimeric protein may be co-expressed with a receptor, particularly an antigen receptor, for example, a CAR or a TCR or a derivative thereof (e.g. a TCR-CAR construct, or single chain TCR construct etc.). The coding sequence for such a receptor may be comprised within a construct as referred to above.

The chimeric protein may also be co-expressed with a safety switch polypeptide, for example as discussed above.

Other polypeptides which may be co-expressed with the chimeric protein include transcription factors, growth factors or other factors which may assist in enhancing functionality of survival of the cell. For example, the transcription factor FOXP3 may be used to maintain the suppressive phenotype of Treg cells. “FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3. A “FOXP3 polypeptide” is a polypeptide having FOXP3 activity i.e. a polypeptide able to bind FOXP3 target DNA and function as a transcription factor regulating development and function of Tregs.

The present nucleic acid molecule or construct may further comprise a nucleic acid sequence encoding a selectable marker. Suitably selectable markers are well known in the art and include, but are not limited to, fluorescent proteins—such as GFP. Suitably, the selectable marker may be a fluorescent protein, for example GFP, YFP, RFP, tdTomato, dsRed, or variants thereof. In some embodiments the fluorescent protein is GFP or a GFP variant.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The use of a selectable marker is advantageous as it allows cells (e.g. Tregs) in which a nucleic acid molecule, construct or vector has been successfully introduced (such that the encoded chimeric protein and any other encoded proteins or polypeptides are expressed) to be selected and isolated from a starting cell population using common methods, e.g. flow cytometry.

In a still further embodiment, the chimeric protein may be co-expressed with a mutant calcineurin protein which is resistant to at least one calcineurin inhibitor, and in particular a mutant calcineurin protein which is resistant to at least one calcineurin inhibitor and sensitive to at least one calcineurin inhibitor. Such calcineurin mutants are discussed further below. In such an embodiment the nucleic acid molecule or construct may further comprise a nucleotide sequence encoding such a mutant calcineurin.

Where two or more coding sequences are expressed from a single nucleic add molecule or construct, they may be linked by a sequence allowing co-expression of the two or more coding sequences. In particular, the co-expression sequence, or alternatively termed, the co-expression site, may enable expression of an encoded protein or polypeptide as a discrete entity. For example, the construct may comprise an internal promoter, an internal ribosome entry sequence (IRES) sequence or a sequence encoding a cleavage site.

In particular the co-expression sequence may encode a self-cleavage sequence in between encoded polypeptides. Particularly, the self-cleaving sequence may be a self-cleaving peptide. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. 2A and 2A-like peptides are believed to cause ribosome skipping, and result in a form of cleavage in which a ribosome skips the formation of peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline.

Suitable self-cleaving domains include P2A, T2A, E2A, and F2A sequences as shown in SEQ ID NOs: 73-77 respectively. The sequences may be modified to include the amino acids GSG at the N-terminus of the 2A peptides. Thus, also included as possible options are sequences corresponding to SEQ ID NOs:. 73-77, but with GSG at the N termini thereof. Such modified alternative 2A sequences are known and reported in the art. Alternative 2A-like sequences which may be used are shown in Donnelly et al (supra), for example a TaV sequence.

The self-cleaving sequences included in the nucleic acid molecule may be the same or different.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site RXXR (SEQ ID NO: 78), for example RRKR (SEQ ID NO: 79).

The nucleic acid molecule/polynucleotides used herein may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

The nucleotide sequence encoding the chimeric protein, and any other coding nucleotide sequences may be provided in a construct in which they are operably linked to a promoter. In some cases, different nucleotide sequences may be operably linked to the same promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. The promoter may be from any source, and may be a viral promoter, or a eukaryotic promoter, including mammalian or human promoters (i.e. a physiological promoter). In an embodiment the promoter is a viral promoter. Particular promoters include LTR promoters, EFS (or functional truncations thereof), SFFV, PGK, and CMV. In an embodiment the promoter is SFFV or a viral LTR promoter. “Operably linked to the same promoter” means that transcription of the polynucleotide sequences may be initiated from the same promoter and that the nucleotide sequences are positioned and oriented for transcription to be initiated from the promoter. Polynucleotides operably linked to a promoter are under transcriptional regulation of the promoter.

A vector is a tool that allows or facilitates the transfer of an entity from one environment to another. As used herein, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide sequence of interest.

The vectors used herein may be, for example, plasmid, mRNA or virus vectors and may include a promoter (as described above) for the expression of a nucleic acid molecule/polynucleotide and optionally a regulator of the promoter.

In an embodiment the vector is a viral vector, for example a retroviral, e.g. a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g. act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (VVPRE), e.g. operably linked to the promoter.

Vectors comprising the present nucleic acid molecules/polynucleotides may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

In some cases, the present nucleic acid molecules may be designed to be used as single constructs which encode the chimeric protein and any other polypeptide (e.g. receptor or marker or other functional polypeptide or protein of interest) and this would be contained in a single vector, it is not precluded that they are introduced into a cell in conjunction with other vectors, for example encoding other polypeptides it may be desired also to introduce into the cell.

As is clear from the above description in addition to the specific polypeptide and nucleotide sequences mentioned herein, also encompassed is the use of variants, or derivatives and fragments thereof.

The term “derivative” or “variant” as used interchangeably herein, in relation to the present proteins or polypeptides includes any substitution of, variation of, modification of, replacement of, deletion of and/or addition of one (or more) amino acid residues from or to the sequence providing that the resultant protein or polypeptide retains the desired function. For example, where the derivative or variant is a signalling domain, the desired function may be the ability of that domain to signal (e.g. activate or inactivate a downstream molecule, where the derivative or variant is a heterodimerization domain, the desired function is interaction with the inducer molecule, e.g. binding to rapamycin or an analogue thereof etc. Alternatively viewed, the variants or derivatives referred to herein are functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 70%, 75%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e. amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

As noted above, the chimeric protein may be co-expressed by a cell in conjunction with a CAR. The term “chimeric antigen receptor” or “CAR” as used herein refers to engineered receptors which can confer an antigen specificity onto cells (for example Tregs). CARs are also known as artificial T-cell receptors, chimeric T-cell receptors or chimeric immunoreceptors. A CAR typically comprises an extracellular domain comprising an antigen-specific targeting region, termed herein an antigen-binding domain, a transmembrane domain, and an endodomain comprising optionally one or more co-stimulatory domains, and an intracellular signaling domain. The antigen-binding domain is typically joined to the transmembrane domain by a hinge domain. The design of CARs, and the various domains that they may contain, is well known in the art.

When the CAR binds its target antigen, this results in the transmission of an activating signal to the cell in which it is expressed. Thus, the CAR directs the specificity of the engineered cells towards the target antigen, particularly towards cells expressing the targeted antigen.

The antigen-binding domain of a CAR may be derived or obtained from any protein or polypeptide which binds (i.e. has affinity for) a desired target antigen, or more generally a desired target molecule. This may be for example, a ligand or receptor, or a physiological binding protein for the target molecule, or a part thereof, or a synthetic or derivative protein. The target molecule may commonly be expressed on the surface of a cell, for example a target cell, or a cell in the vicinity of a target cell (for a bystander effect), but need not be. Depending on the nature and specificity of the antigen binding domain, the CAR may recognise a soluble molecule, for example where the antigen-binding domain is based on, or derived from, a cellular receptor.

The antigen-binding domain is most commonly derived from antibody variable chains (for example it commonly takes the form of a scFv), but may also be generated from T-cell receptor variable domains or, as mentioned above, other molecules, such as receptors for ligands or other binding molecules.

The CAR is typically expressed as a polypeptide also comprising a signal sequence (also known as a leader sequence), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

The antigen-binding domain provides the CAR with the ability to bind a predetermined antigen of interest. The antigen-binding domain preferably targets an antigen of clinical interest or an antigen at a site of disease.

As noted above, the antigen-binding domain may be any protein or peptide that possesses the ability to specifically recognize and bind to a biological molecule (e.g., a cell surface receptor or a component thereof). The antigen-binding domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

The term “directed against” is synonymous with “specific for” or “anti”. Put another way, the CAR recognises a target molecule. Accordingly, it is meant that the CAR is capable of binding specifically to a specified or given antigen, or target. In particular, the antigen-binding domain of the CAR is capable of binding specifically to the target molecule or antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing the target molecule or antigen, particularly a target cell expressing the target antigen or molecule on its cell surface.

Antigens which may be targeted by the present CAR include, but are not limited to, antigens expressed on cells associated with transplanted organs, autoimmune diseases, allergic diseases and inflammatory diseases (e.g. neurodegenerative disease). It will be understood by a skilled person that where the cell engineered to express the CAR is a Treg cell, or a precursor therefor, due to the bystander effect of Treg cells, the antigen may be simply present and/or expressed at the site of transplantation, inflammation or disease.

Antigens expressed on cells associated with neurodegenerative disease include those presented on glial cells, e.g. MOG. Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

In an embodiment the CAR is directed against an HLA antigen, and in particular an HLA-A2 antigen.

Antibodies against such antigens and are known in the art, and conveniently a scFv may be obtained or generated bases on a known or available antibody. In this regard VH and VL, and CDR sequences are publically available to aid the preparation of such an antibody-binding domain, for example in WO 2020/044055, the disclosure of which is herein incorporated by reference. Any of the antigen binding domains, or CDR, VH, and/or VL sequences disclosed in WO 2020/044055 may be used.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more regions or epitopes within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

Engineered cells may be generated by introducing a nucleic acid molecule, construct, or vector as defined herein, by one of many means including transduction with a viral vector, and transfection with DNA or RNA.

The present cell may be made by: introducing to a cell (e.g. by transduction or transfection) the nucleic acid molecule, construct or vector as defined herein.

Suitable cells are discussed further below, but the cell may be from a sample isolated from a subject. The subject may be a donor subject, or a subject for therapy (i.e. the cell may be an autologous cell, or a donor cell, for introduction to another recipient, e.g. an allogeneic cell).

The cell may be generated by a method comprising the following steps:
(i) isolation of a cell-containing sample from a subject or provision of a cell-containing sample; and
(ii) introduction into (e.g. by transduction or transfection) the cell-containing sample of a nucleic acid molecule, construct, or vector as defined herein, to provide a population of engineered cells.

A cell into which a nucleic acid molecule, construct or vector is to be introduced may be referred to as a target cell. A target cell-enriched sample may be isolated from, enriched, and/or generated from the cell-containing sample prior to and/or after step (ii) of the method. For example, isolation, enrichment and/or generation of Tregs (or other target cells)_may be performed prior to and/or after step (ii) to isolate, enrich or generate a Treg-enriched sample. Isolation and/or enrichment from a cell-containing sample may be performed after step (ii) to enrich for cells and/or Tregs (or other target cells) comprising the CAR, the nucleic acid molecule/polynucleotide, the construct and/or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching other target cells are known in the art.

The target cell may be a Treg cell, or precursor or a progenitor therefor.

An “engineered cell” means a cell which has been modified to comprise or express a polynucleotide which is not naturally encoded by the cell. Methods for engineering cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation, as discussed above. Any suitable method may be used to introduce a nucleic acid sequence into a cell. Non-viral technologies such as amphipathic cell penetrating peptides may be used to introduce nucleic acid.

Accordingly, the nucleic acid molecule as described herein is not naturally expressed by a corresponding, unmodified cell. Indeed, the nucleic acid molecule encoding the chimeric protein is an artificial construct, and could not occur or be expressed naturally. Suitably, an engineered cell is a cell which has been modified e.g. by transduction or by transfection. Suitably, an engineered cell is a cell which has been modified or whose genome has been modified e.g. by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign nucleic acid, e.g. DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing a nucleic acid as described herein by one of many means including transduction with a viral vector, transfection with DNA or RNA. Cells may be activated and/or expanded prior to, or after, the introduction of a nucleic acid as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g. Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The cell may be an immune cell, or a precursor therefor. A precursor cell may be a progenitor cell. Representative immune cells thus include T-cells, in particular, cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, dendritic cells, MDSC, neutrophils, and macrophages. Precursors of immune cells include pluripotent stem cells, e.g. induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL) or a Treg cell.

In particular, the immune cell may be a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

A T cell as used herein is a lymphocyte including any type of T cell, such as an alpha beta T cell (e.g. CD8 or CD4+), a gamma delta T cell, a memory T cell, a Treg cell.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tcon) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs as referred to herein may proliferate 5%, 10%, 20%, 30%, 40%, 50%, 60%, 70%, 90%, 95% or 99% less than the same Tconv cells cultured in the absence of the Tregs. For example, antigen-specific Tconv cells co-cultured with the present Tregs may proliferate 5%, 10%, 20%, 30%, 40%, 50%, 60%, 70%, 90%, 95% or 99% less than the same Tconv cells cultured in the presence of non-engineered Tregs. The cells comprising the nucleic acid, expression construct or vector as defined herein, e.g. Tregs may have an increased suppressive activity as compared to non-engineered Tregs (e.g. an increased suppressive activity of at least 5, 10, 20, 30, 40, 50, 60, 70, 80 or 90%). Antigen-specific Tconv cells co-cultured with the Tregs herein may express at least 10%, at least 20%, at least 30%, at least 40%, at least 50%, or at least 60% less effector cytokine than corresponding Tconv cells cultured in the absence of the Tregs (e.g. in the presence of non-engineered Tregs). The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural T regs are CD4+CD25⁺FOXP3⁺Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating FOXP3 expression (Polansky, J. K., et al., 2008. European journal of immunology, 38(6), pp.1654-1663).

Further suitable Tregs include, but are not limited to, Tr1 cells (which do not express FOXP3, and have high IL-10 production); CD8+FOXP3+ T cells; and γδ FOXP3⁺ T cells.

Different subpopulations of Tregs are known to exist, including naïve Tregs (CD45RA⁺FOXP3^{low}), effector/memory Tregs (CD45RA⁻FOXP3^{high}) and cytokine-producing Tregs (CD45RA⁻FOXP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA+. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4+non-regulatory or Tcon cell from the same subject or donor. Particularly, naïve Tregs may express less than 90, 80, 70, 60, 50, 40, 30, 20 or 10% CD127 as compared to a CD4+non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered cell is to be administered. Suitably, the subject to be treated is a mammal, particularly a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered cell is to be administered.

Suitably, the Treg is part of a population of cells. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. A nucleic acid molecule, construct or vector as described herein may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference).

As used herein, the term “conventional T cell” or Tcon or Tconv (used interchangeably herein) means a T lymphocyte cell which expresses an af3 T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing the nucleic acid which includes a sequence coding for FOXP3 Alternatively, the engineered Treg may be generated from a Tcon by in vitro culture of CD4+ CD25-FOXP3− cells in the presence of IL-2 and TGF-β.

When the chimeric protein is induced, a Treg herein may have increased persistence as compared to a Treg cell without the inducible chimeric protein. “Persistence” as used herein defines the length of time that Tregs can survive in a particular environment, e.g. in vivo (e.g. in a human patient or animal model). A Treg as disclosed herein may have at least 10, 20, 30, 40, 50, 60, 70, 80 or 90% increased persistence as compared to a Treg which does not express the chimeric protein herein, or a Treg which is not induced. Persistence can be measured by for example, determining the amount or numbers of administered cells within a subject or patient over time, where cells expressing a chimeric protein of the invention are compared to equivalent cell types which do not express the chimeric protein, or compared to non-engineered cells. It is possible to track administered cells, for example, using a marker protein, e.g. CD34 for cells which also express a RQR8 safety switch as part of the chimeric protein or as a separate molecule.

In another embodiment the target cell into which the nucleic acid molecule, construct or vector is introduced is not a cell intended for therapy. In an embodiment the cell is a production host cell. The cell may be for production of the nucleic acid, e.g. cloning, or vector, or polypeptides.

Also provided herein is a cell population comprising a cell as defined or described herein. It will be appreciated that a cell population may comprise both the present cells comprising a nucleic acid molecule, construct or vector as defined herein, and cells which do not comprise the nucleic acid molecule, construct or vector, e.g. untransduced or untransfected cells. Although in a particular embodiment, all the cells in a population may comprise the nucleic acid, expression construct or vector, cell populations having at least 10, 20, 30, 40, 50, 60, 70, 80, 90, 95 or 99% of cells comprising a nucleic acid, expression construct or vector are provided.

As indicated above, the chimeric protein may be co-expressed by a cell in conjunction with a mutant calcineurin which is resistant to at least one calcineurin inhibitor (CNI), In particular, the mutant calcineurin may be resistant to one calcineurin inhibitor and sensitive to another calcineurin inhibitor. Such calcineurin mutants are described in Brewin et al., 2009, Blood, 114(23), 4792-4802, incorporated herein by reference. Calcineurin inhibitors, notably FK506 (tacrolimus) and cyclosporine (CsA) are immunosuppressants commonly used in ACT. Thus, by co-expressing such a calcineurin mutant in a cell for ACT, the cell may be rendered resistant to the immunosuppressant. This may confer various advantages on the cell. For example, such co-expression may advantageously allow the ACT subject (i.e. cell recipient) to continue immunosuppressant therapy, and may allow lymphodepletion of the recipient to be avoided. Thus it may not be necessary to interrupt or stop administering an immunosuppressant to the subject. Further, advantageously, co-expressing such a mutant in the cell along with the chimeric protein (and any other protein of interest, such as CAR), may allow for selective expansion of transduced (or transfected etc.) cells. In this regard, during in vitro expansion protocols the transduced or transfected cell population (that is the cells resulting from the transduction/transfection protocol etc.), may be cultured in the presence of the calcineurin inhibitor to which the cells have been rendered resistant by virtue of the mutant calcineurin. Any non-transduced/non-transfected cells which are undesired in the final cell preparation for ACT will not be able to grow (proliferate) in the presence of the calcineurin inhibitor and will not be expanded. Further, if the mutant calcineurin is resistant to a particular inhibitor, but sensitive to another, then that provides a safety mechanism by which the cell containing the mutant calcineurin may be controlled or eliminated—by administering the inhibitor to which the mutant calcineurin is sensitive.

As indicated above, such mutant calcineurin proteins are known in the art. In particular, the mutant calcineurin may be resistant to FK506 but sensitive to CsA, resistant to CsA but sensitive to FK506, or resistant to both FK506 and CsA. Such mutants are described in Brewin et al., 2009 (supra).

The mutant calcineurin may comprise mutations in the A subunit (CNa) (SEQ ID NO: 97) or the B subunit (CNb) (SEQ ID NO: 98) of calcineurin.

Mutations to the A subunit may comprise a mutation at one or more of the following positions with reference to SEQ ID NO. 97; V314, Y341, M347, T351, W352, S353, L354, F356 and K360.

Particularly, a mutated A subunit may comprise one or more of the following mutations with reference to SEQ ID NO: 97:
V314K, V314R or V314F;
Y341F;
M347W, M347R or M347E;
T351E;
W352A, W352C or W352E;
S353H or S353N;
L354A;
F356A; and/or
K360A or K360F.

Combinations of mutations that may be made to the A subunit include with respect to SEQ ID NO. 97:
L354A and K360A
L354A and K360F
T351E and K360F
W352A and S353H
T351E and L354A
W353C and K360F
W352C, L354A and K360F
V314K and Y341F
V314R and Y341F

Mutations to the B subunit may be at one or more of the following positions with reference to SEQ ID NO: 98; Q51, L116, M119, V120, G121, N122, N123, L124, K125 and K165.

Particularly, a mutated B subunit may comprise one or more of the following mutations with reference to SEQ ID NO: 98;
Q515
L116R or L116Y
M119A, M119Wor M119-F-Ins
V120L, V1205, V120D or V120F
G121-LF-Ins
N122A, N122H, N122F, or N122S
N123H, N123R, N123F, N123K, or N123W
L124T
K125A, K125E, K125W, K125-LA-Ins, K125-VQ-Ins or K125-1E-Ins; and
K165Q.

Combinations of mutations that may be made to the B subunit include with respect to SEQ ID NO: 98;
V1205 and L124T;
V120D and L124T
N123W and K125-LA-Ins
L124T and K125-LA-Ins
V120D and K125-LA-Ins and
M119-F-Ins and G121-LF-Ins

Particular mention may be made of the following mutants:
(i) CNa12 which is resistant to FK506, but sensitive to CsA. This mutant comprises mutations T351E and L354A. The sequence of CNa12 is shown in SEQ ID NO: 94;
(ii) CNa18, CNa19, CNa20, CNa21 and CNa22 and CNb26 which are resistant to CsA, but sensitive to FK506. These mutants comprise, respectively, mutations M347E; V314K; V314R; Y3F, V314K and Y341F; V314R and Y341F; and V1205 and L124T. The sequence of CNa22 is shown in SEQ ID NO: 95;
(iii) CNb21, CNb23, and CNb30 which are resistant to both CsA and FK506. These mutants comprise, respectively, mutations K125-VQ-Ins; K125-LA-Ins; and L124T and K125-LA-Ins. The sequence of CNb30 is shown in SEQ ID NO: 96.

Accordingly, in an embodiment, a mutant calcineurin for use herein comprises an amino acid sequence as shown in any one of SEQ ID NOs: 94, 95 or 96 or an amino acid sequence which has at least 85, 90, 95, 97, 98 or 99% sequence identity to SEQ ID NO: 94, 95 or 96, provided that the mutations indicated above for SEQ NOs: 94, 95 or 96 are retained.

Other mutants are described in Brewin (supra) and may also be used. The skilled person would be able to derive new mutants comprising other mutations in addition or in place of those disclosed in Tables 1 and 2 of Brewin, e.g. different substitutions, or different combinations of the mutations which are disclosed.

Also disclosed in Brewin 2009 are assays for screening for resistance or susceptibility to FK506 or CsA which may be used to select mutants. The terms “sensitive” and “susceptible” are used interchangeably herein. A mutant which is resistant to a given CNI means a mutant which exhibits resistance to the CNI, that is a mutant which is able to confer on a cell expressing it the ability to grow, or proliferate, or to exhibit a functional property in its presence. In particular, a cell comprising the mutant may exhibit increased growth, which may be reflected in an increase in a functional property, for example increased IL-2 secretion in Jurkat cells expressing the mutant, compared to a cell which does not comprise the mutant.

It is not required that a mutant exhibits 100% resistance to a given CNI. The levels of resistance may vary, and some degree of susceptibility may be tolerated, as long as there is an observable increase in growth or functionality of cells expressing the mutant in the presence of the CNI in question, e.g. as compared to cells without (not expressing) the mutant. For example, the mutant may exhibit at least 35%, 40 or 45% resistance, more particularly at least 50, 55, 60 or 65% resistance.

As discussed above, a particular type of cell of interest is a Treg cell. Treg cells are administered in ACT to suppress undesired immune response or immune activity. The expression of mutant calcineurins in Treg cells is of particular interest.

Accordingly in a further aspect, there is more generally provided herein a Treg cell which comprises a mutant calcineurin which is resistant to at least one calcineurin inhibitor.

In an embodiment of this aspect, the mutant calcineurin is resistant to at least one calcineurin inhibitor and sensitive to at least one calcineurin inhibitor. In particular the mutant is (i) resistant to FK506 and sensitive to CsA or (ii) resistant to CsA and sensitive to FK506. The mutant may be any one of the mutants discussed above.

The Treg cell may be engineered or modified to comprise the mutant calcineurin by introducing a nucleic acid, construct or vector comprising a nucleotide sequence encoding the mutant calcineurin. Such a Treg cell may further comprise or express another protein of interest. This may be, for example, a chimeric protein as described herein and/or a CAR or other chimeric receptor, a TCR, or any other polypeptide as referred to or described herein.

Particularly, a cell, such as a Tcell, e.g., a Treg cell, may further comprise or express a chimeric protein which is capable of providing a STAT, e.g. STAT5 mediated signal to the cell. Such a chimeric protein may be one as described herein, or may be an alternative molecule, such as one described in WO2020/044055, which is incorporated by reference. Particularly, such a chimeric protein may comprise a JAK1 and/or JAK2 binding domain and a STAT5 association motif, as described in detail above. Thus, in this regard, a cell, e.g., a Treg cell is provided which comprises a nucleic acid molecule comprising a nucleotide sequence encoding a mutated calcineurin and a nucleic acid molecule comprising a nucleotide sequence encoding a chimeric protein comprising a JAK1 and/or JAK2 binding domain and a STAT, particularly a STAT5 association motif, particularly wherein said chimeric protein is capable of dimerization (e.g. a chimeric protein of the invention). In one embodiment, the JAK1 and/or JAK2 binding domain and STAT5 association motif may be present within the endodomain of a chimeric antigen receptor, e.g. as described in WO2020/044055. As discussed above, the chimeric protein should be capable of providing a STAT, e.g. a STAT5 mediated signal to the cell (e.g. upon dimerization which may be constitutively provided for or inducible, e.g. by binding of the chimeric protein to a target or to a ligand or CID as discussed herein). The cell may also in this embodiment comprise a CAR as defined herein.

Thus, the mutant calcineurins may be used in Treg cells which have been, are being, or will be engineered for another purpose, e.g. to express another desired heterologous (i.e. non-native) protein.

Another aspect provided herein is a method for selectively expanding engineered Treg cells, said method comprising introducing into a Treg cell a nucleic acid molecule, construct or vector comprising a nucleotide sequence encoding a mutant calcineurin as defined herein, and culturing said cells in the presence of a calcineurin inhibitor to which the mutant calcineurin is resistant. Particularly, the cell may further comprise a chimeric receptor comprising a JAK1 and/or JAK2 binding domain and a STAT, e.g. a STAT5 association motif.

The engineered Treg cell may comprise a further nucleotide sequence which is, or has been, introduced into the cell, and which encodes a protein of interest.

The culturing step may comprise any known or desired protocol for activating and/or expanding cells. For example, the cells may be grown in the presence of CD3 and/or CD28 as described above.

There is also provided a pharmaceutical composition comprising a cell or cell population as defined or described herein, a vector as defined herein. The vector may be used for gene therapy. Thus, rather than administering a cell, a vector may be administered instead, to modify endogenous cells in the subject to express the introduced nucleic acid molecule. Vectors suitable for use in gene therapy are known in the art, and include viral vectors.

A pharmaceutical composition is a composition that comprises or consists of a therapeutically effective amount of a pharmaceutically active agent i.e. the cell (e.g. Treg), cell population or vector. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The cell, cell population or pharmaceutical composition as described herein can be administered parenterally, for example, intravenously, or they may be administered by infusion techniques. The cell, cell population or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly.

The preparation of suitable parenteral formulations under sterile conditions is readily accomplished by standard pharmaceutical techniques well-known to those skilled in the art.

The pharmaceutical compositions may comprise cells in infusion media, for example sterile isotonic solution. The pharmaceutical composition may be enclosed in ampoules, disposable syringes or multiple dose vials made of glass or plastic.

The cell, cell population or pharmaceutical composition may be administered in a single or in multiple doses. Particularly, the cell, cell population or pharmaceutical composition may be administered in a single, one off dose. The pharmaceutical composition may be formulated accordingly.

The pharmaceutical composition may further comprise one or more active agents. The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, I L18).

Depending upon the disease/condition and subject to be treated, as well as the route of administration, the cell, cell population or pharmaceutical composition may be administered at varying doses (e.g. measured in cells/kg or cells/subject).

The physician in any event will determine the actual dosage which will be most suitable for any individual subject and it will vary with the age, weight and response of the particular subject. Typically, however, for the cells herein, doses of 5×10⁷ to 3×10⁹ cells, or 10⁸ to 2×10⁹ cells per subject may be administered.

The cell may be appropriately modified for use in a pharmaceutical composition. For example, cells may be cryopreserved and thawed at an appropriate time, before being infused into a subject.

Further provided herein is the use of kits, or combination products, comprising the cell, cell population and/or pharmaceutical composition herein. Preferably said kits are for use in the methods and uses as described herein, e.g., the therapeutic methods as described herein. Preferably said kits comprise instructions for use of the kit components. Kits or compositions may further comprise the CID, e.g. rapamycin or an analogue thereof.

The cells, cell populations, compositions and vectors herein may be for use therapy, that is in treating or preventing a disease or condition. As noted above, the cell by which the chimeric protein is expressed is typically a cell which is modified, or engineered to express a further molecule (e.g. a further protein), notably a receptor, e.g. a CAR or TCR, Accordingly, the therapy may be for the prevention or treatment of a disease or condition which may be treated by or with cell expressing the receptor, e.g. the CAR. The cells and compositions containing them are for adoptive cell therapy (ACT). Various conditions may be treated by administration of cells, including particularly Treg cells, expressing a CAR according to the present disclosure. As noted above, this may be conditions responsive to immunosuppression, and particularly the immunosuppressive effects of Tregs cells. The cells, cell populations, compositions and vectors described herein may thus be used for inducing, or achieving, immunosuppression in a subject. The Treg cells administered, or modified in vivo, may be targeted by expression of the receptor, e.g. CAR. Conditions suitable for such treatment include infectious, neurodegenerative or inflammatory disease, or more broadly a condition associated with any undesired or unwanted or deleterious immune response.

Conditions to be treated or prevented include inflammation, or alternatively put, a condition associated with or involving inflammation. Inflammation may be chronic or acute. Furthermore, the inflammation may be low-level or systemic inflammation. For example the inflammation may be inflammation which occurs in the context of a metabolic disorder, for example metabolic syndrome, or in the context of insulin resistance, or type II diabetes or obesity and such like.

In particular, the cells, cell populations, vectors and pharmaceutical compositions provide a means for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation. The cells, cell populations, vectors and pharmaceutical compositions may be used in a method which comprises the step of administering a cell, cell populations, vector or a pharmaceutical composition as described herein to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the engineered cells, e.g. Tregs, may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered cells, e.g. Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the cells may express a CAR which comprises an antigen binding domain which is capable of specifically binding to a HLA antigen that is present in the graft (transplant) donor but not in the graft (transplant) recipient.

Suitably, the transplant is a liver transplant. In embodiments where the transplant is a liver transplant, the antigen may be a HLA antigen present in the transplanted liver but not in the patient, a liver-specific antigen such as NTCP, or an antigen whose expression is up-regulated during rejection such as CCL19, MMP9, SLC1A3, MM P7, HMMR, TOP2A, GPNMB, PLA2G7, CXCL9, FABPS, GBP2, CD74, CXCL10, UBD, CD27, CD48, CXCL11.

As discussed above, in one representative and preferred embodiment the antigen is HLA-A2.

A method for treating a disease or condition relates to the therapeutic use of the cells herein. In this respect, the cells may be administered to a subject having an existing disease or condition in order to lessen, reduce or improve at least one symptom associated with the disease or condition and/or to slow down, reduce or block the progression of the disease.

Suitably, treating and/or preventing cellular and/or humoral transplant rejection may refer to administering an effective amount of the cells (e.g. Tregs) such that the amount of immunosuppressive drugs that a transplant recipient requires is reduced, or may enable the discontinuation of immunosuppressive drugs.

Preventing a disease or condition relates to the prophylactic use of the cells herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example, Amyotrophic Lateral Sclerosis (ALS); Chronic inflammatory demyelinating polyneuropathy (CI PD) and juvenile onset diabetes.

As indicated above, the chimeric protein is not limited to use in the context of immunosuppressive therapy, and the protein may be expressed in cells for the treatment of conditions such as cancer or infections. It may be desirable in such contexts to kill or ablate cancer or infected cells, and in such cases the chimeric protein may be expressed in cytotoxic cells, such as cytotoxic T cells or NK cells, or precursors therefor. The receptor (e.g. CAR or TCR) co-expressed with the chimeric protein in such cases may be directed against a cancer antigen or an antigen from a pathogen etc.

The medical use of or method herein may involve the steps of:
(i) isolating a cell-containing sample or providing a cell-containing sample;
(ii) introducing a nucleic acid molecule, construct or a vector as defined herein to the cell; and
(iii) administering the cells from (ii) to a subject.

The cell may be a Treg as defined herein. An enriched Treg population may be isolated and/or generated from the cell containing sample prior to, and/or after, step (ii) of the method. For example, isolation and/or generation may be performed prior to and/or after step (ii) to isolate and/or generate an enriched Treg sample.

Enrichment may be performed after step (ii) to enrich for cells and/or Tregs comprising the chimeric protein, the nucleic acid molecule, construct, and/or the vector as described herein.

Suitably, the cell may be autologous. Suitably, the cell may be allogenic. Suitably, the cell (e.g. the engineered Treg) may be administered is combination with one or more other therapeutic agents, such as lympho-depletive agents. The engineered cell, e.g. Treg, may be administered simultaneously with or sequentially with (i.e. prior to or after) the one or more other therapeutic agents.

Cells, e.g. Tregs, may be activated and/or expanded prior to, or after, the introduction of a nucleic acid molecule as described herein, for example by treatment with an anti-CD3 monoclonal antibody or both anti-CD3 and anti-CD28 monoclonal antibodies. Expansion protocols are discussed above.

The cell, e.g. Tregs, may be washed after each step of the method, in particular after expansion.

The population of engineered cells, e.g., Treg cells may be further enriched by any method known to those of skill in the art, for example by FACS or magnetic bead sorting.

The steps of the method of production may be performed in a closed and sterile cell culture system.

The invention may also provide a method for increasing the stability and/or suppressive function of a cell comprising the step of introducing a nucleic acid molecule, an expression construct or vector as provided herein into the cell. An increase in suppressive function can be measured as discussed above, for example by co-culturing activated antigen-specific Tconv cells with cells of the invention, and for example measuring the levels the cytokines produced by the Tconv cells. An increase in suppressive function may be an increase of at least 10, 20, 30, 40, 50, 60, 70, 80, or 90% as compared to a non-engineered or non-induced Treg.

An increase in stability of a cell, e.g. a Treg as defined herein, refers to an increase in the persistence or survival of those cells or to an increase in the proportion of cells retaining a Treg phenotype over a time period (e.g. to cells retaining Treg markers such as FOXP3 and Helios) as compared to a non-engineered or non-induced Treg. An increase in stability may be an increase in stability of at least 10, 20, 30, 40, 50, 60, 70, 80 or 90%, and may be measured by techniques known in the art, e.g. staining of Treg cell markers within a population of cells, and analysis by FACS.

A further aspect provided herein is a combination product comprising (a) a cell, cell population, vector or pharmaceutical composition as defined herein, and (b) a CID, for use in therapy, particularly ACT or gene therapy. The therapy may be any therapy as defined above, and further described herein.

The components (a) and (b) of the combination product may be for separate, sequential or simultaneous use.

The components (a) and (b) of the combination product will typically be provided as separate compositions, i.e. they will be formulated separately. Thus, the combination product may alternatively be defined or referred to as a kit.

The components (a) and (b) will be administered to the subject separately. They may be administered to a subject at the same time, or at different times, for example at spaced apart time intervals. For example, the cells etc may be administered first, followed by the CID. The CID may be administered after an appropriate time interval, or shortly after the cells etc. The skilled clinician would readily be able to devise an appropriate administration regime according to principles known in the art.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure. It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. All publications mentioned herein are incorporated herein by reference.

The invention will now be further described by way of Examples, which are meant to serve to assist one of ordinary skill in the art in carrying out the invention and are not intended in any way to limit the scope of the invention.

### Example 1

### Materials and Methods

### Cloning:

A construct of the invention was designed in house (SEQ ID NO: 86) comprising an exo domain and TM of RQR8 (safety switch), FRB and FKBP12 intracellular binding domain and a truncated IL2RB signalling domain, for manufacturing. Cloning may be carried out into the pMP71 backbone and D5a high efficiency bacteria may be transformed with plasmid and grown with the selection agent ampicillin. DNA may be extracted using a Miniprep Kit (Qiagen). Inserts can be transferred into a lentiviral backbone by PCR cloning.

### Collection of PBMCs:

Leukocyte cones can be supplied by NHS blood and transplant. PBMC may be isolated using a density centrifugation protocol. Briefly, blood may be diluted 1:1 with 1×PBS and layered over Ficoll-Paque (GE Healthcare). Samples may be centrifuged and the leukocyte layer removed and washed in PBS.

### Treg and Tconv Isolation Protocol:

Blood cones may be used to derive Treg and Teff populations. Blood cones may be subjected to CD4 enrichment via negative selection using RosetteSep™ Human CD4+ T Cell Enrichment Cocktail. Subsequently, CD4+ cells may be isolated using density centrifugation. CD4+ CD25+ T cells may then be isolated via positive selection using CD25 microbeads II (Miltenyi). The CD4+ CD25− fraction of cells may be retained to serve as conventional T cell (Tconv) populations. The CD4+ CD25+ fraction may be stained with flow cytometry antibodies CD4 FITC (OKT4, Biolegend), CD25 PE-Cy7 (BC96, Biolegend), CD127 BV421 (A019D5, Biolegend), CD45RA BV510 (HI100, Biolegend) and the LIVE/DEAD™ Fixable Near-IR - Dead Cell Stain (Thermofisher) before FACS sorting. Where indicated, CD4+ CD25+ CD127low (Bulk Tregs) or CD4+ CD25+ CD127low CD45RA+ (CD45RA+ Tregs) may be sorted and used.

### T Conv Culture Media:

Human Tconv may be grown in RPMI-1640 (Gibco) supplemented with 10% heat inactivated foetal bovine serum; penicillin; streptomycin; L-glutamine (Gibco).

### T Reg Culture Media and Expansion:

Human Regulatory T cells may be cultured in Texmacs media (Miltenyi) supplemented with IL-2 and activated with Human T-Activator CD3/CD28 Dynabeads™ (Gibco). Cells may be re-fed every 2 to 3 days with Treg culture media supplemented with IL-2. A second round of stimulation with Dynabeads™ may be performed to promote further expansion of Treg cells.

### Transfection and Viral Particle Production

### Lentivirus:

HEK293T cells may be seeded and cultured in DMEM (Dulbecco's Modified Eagle's Medium) +10% Fetal Bovine Serum (FBS) for 24 hours. Transfection reagents may be brought to room temperature and mixed with DNA construct/plasmid of interest, packaging plasmid (pD8.91) and viral envelope (pVSV-G). PEI may be added to the diluted DNA and mixed and added to HEK293Ts. Supernatant may be harvested 48 hours post-transfection, filtered and virus concentrated.

### Transduction of T Cells

Tconv may be activated with anti-CD3 and anti-CD28 Dynabeads (Gibco) and resuspended in T cell culture media. Non-tissue culture-treated 24-well plates may be prepared by coating with Retronectin (Takahara-bio—Otsu, Japan), cell suspension may be added together with lentiviral supernatant. Cells may be incubated and media exchange may be performed on alternate days. Cells may be used for experiments 7 days post-transduction.

### Flow Cytometry Staining to Determine RQR8 Expression

T cells may be removed from culture and washed in FACS buffer and stained with the LIVE/DEAD™ Fixable Near-IR—Dead Cell Stain (Thermofisher) in PBS first and then with anti-CD4 AF700 (RPA-T4, BD), anti-CD34 FITC (QBEND/10, Thermofisher) and anti-CD3 PE-Cy7 in FACS staining buffer. For intracellular staining of FOXP3, cells may be fixed and permeabilized and stained with the anti-FOXP3 PE (150D/E4, Thermofisher) antibody. Cells may be analysed on a BD LSRII flow cytometer.

### Flow Cytometry Phenotyping oOf Transduced Cells

T cells may be removed from culture and stained for live cells using LIVE/DEADTM Fixable Near-IR - Dead Cell Stain as described above. Surface staining of the cells may be performed using anti-CD4 AF700, anti-CD25 PE-Cy7 (BC96, Biolegend), anti-CD39 PerCPCy5.5 (A1, Biolegend), anti-CD62L PE-CF594 (DREG-56, BD), anti-TIM3 BV786 (7D3, BD), anti-TIGIT BV605 (A15153G, Biolegend), anti-CD45R0 BUV395 (UCHL1, BD), anti-CD279 BUV737 (EH12.1, BD) and anti-CD223 BV711 (11C3C65, Biolegend). Cells may be permeabilised and stained with anti-Foxp3 PE (150D/E4, Thermofisher).

### Data Analysis

Flow cytometric data was analysed using the Flow Cytometry Analysis software FlowJo (Flowjo,LLC). All statistical Analysis were performed using Graphpad Prism v.5 (Graphpad, Software)

### Example 1a

### Expression in Jurkats

Constructs (e.g. SEQ ID NO: 86) may be cloned into a lentiviral backbone encoding a puromycin resistance gene, as described above. Viral vectors were produced and used for the transduction of the Jurkat T cell line. Two days after transduction, Jurkat cells were selected with 4 μg/ml puromycin for one week. Cells may be counted and 0.5*10 6 cells may be stained with an anti-CD34 antibody as described above to determine the level of chimeric protein expressed in the cells. Expression may be assessed by flow cytometry as described above.

### Example 2

### STAT5 Signaling of Constructs

Different constructs of the chimeric protein are cloned into a lentiviral backbone encoding a puromycin resistance gene. Viral vectors are produced and used for the transduction of a NFAT, NfkB and STAT5 Jurkat reporter cell line. Here, the N FAT, NfkB or STAT5 response element control the activity of a luc2 reporter gene. Two days after transduction, Jurkat cells are selected with 4 μg/mIpuromycin for one week. Cells are then activated using rapamycin and eight hours after activation luciferase is assessed in the different reporter cell lines using ONE-Glo™ Luciferase Assay System (Promega).

### Example 3

### Generation of Regulatory T Cells Expressing the Chimeric Protein

Regulatory T cells are purified and FACS sorted as CD4+ CD25+ CD127− cells from healthy donors. Cells are activated using Human T-Activator CD3/CD28 Dynabeads™ (ThermoFisher Scientific) in X-Vivo medium (Lonza) in the presence of Interleukin-2 (1000 IU/mI). After 48 hours of activation, cells are transduced with lentiviral particles, encoding the chimeric protein constructs. Cells are further expanded, and expansion rate is compared between the different conditions. At day 14 cells are harvested and counted. 0.5*106 cells are stained with an anti-CD34 antibody. The level of RQR8 expression and transduction efficiency was assessed by Flow Cytometry looking at the percentage of anti-CD34 antibody. The Treg phenotype is assessed by surface staining with anti-CD4, anti-CD25, anti-CD127, anti-CD8, anti-GITR, anti-CD39, anti-CD45RA, anti-CD45RO, anti-ICOS and intracellular staining with anti-FOXP3 and anti-HELIOS, following fixation and permeabilization (Transcription Factor Staining Buffer Set, ThermoFisher Scientific)

### Example 4

### STAT5 Phosphorylation Analysis as an Indicator of IL2R Signaling

Transduced Tregs with chimeric protein were rested overnight in culture media without I L2. STAT5 phosphorylation of Tregs was assessed by FACS analysis 10 and 120 minutes after culture with media alone, or rapamycin.

### Example 5

### Cloning and Expression of Constructs in Cells

### Materials and Methods

### Cloning:

Two representative constructs were designed in house as shown in FIGS. 3, and whole sequences were codon optimized for expression in human cells. Constructs were cloned into a Lentiviral backbone (pcLV) which included the GFP reporter gene. These constructs were transformed into Stble3 bacteria with the selection agent Kanamycin. Colonies were picked and DNA was extracted using a Mini-prep kit (Promega). DNA was checked and confirmed using Sanger sequencing and correct clones were amplified and purified using a Maxi-prep kit (Qiagen). The final 2 constructs were designated pQTX-0353 (SEQ ID NO: 99) and pQTX-0354 (SEQ ID NO: 100). Both constructs comprise the SSFV (spleen focus forming virus) promoter, followed by a truncated safety switch sequence ARSR8, comprising a TCRβ signal peptide (21 aa), and two CD20 mimotope sequences (9 aa each) separated by a linker (6 aa). The ARSR8 sequence is linked via a 6 aa (pQTX-0353) or 17 aa (pQTX-354) linker to: a FRB dimerization domain (94 aa); 5 aa linker; a FKBP dimerization domain (107 aa); 17 aa linker; IL2Rβ transmembrane domain (25 aa); IL2Rβ. Y510 signalling domain (94 aa); P2A self-cleavage peptide (19 aa); eGFP reporter (238 aa). A MIul restriction enzyme site (TR) is present between the signaling domain and the P2A sequence.

### Transfection and Lentiviral Particle Production:

HEK293T/17 cells were seeded and cultured in DM EM (Dulbecco's Modified Eagle's Medium)+10% FBS (Fetal Bovine Serum) for 24 hours. Fugene HD (Promega) transfection reagent was brought to room temperature and mixed with the construct DNA, packaging plasmid (pD8.91) and envelope plasmid (pVSV-G). This mixture was incubated for 10 minutes and then added dropwise to the HEK293T/17 cells. Supernatant was harvested 48 hours post-transfection, sterile filtered and concentrated via ultra-centrifugation.

### Isolation, Transduction and Expansion of Human Treg Cells:

CD45RA⁺ human Tregs were isolated from healthy donor blood. Tregs were transduced with various constructs using the required multiplicity of infection (MOI). Non-tissue culture treated well plates were coated with RetroNectin® (Takara Bio, Shiga, Japan) prior to addition of viral particles and Tregs followed by spinoculation. Tregs were expanded using Dynabeads™ Human T-Activator CD3/CD28 for T Cell Expansion and Activation (ThermoFisher Scientific, Massachusetts, USA), X-VIVO™ 15 media (Lonza, Basel, Switzerland) supplemented with 5% heat-inactivated human serum (Merck Life Science UK Limited, Dorset, UK) and interleukin-2 (IL-2; Proleukin®, Clinigen, Burton upon Trent, UK). IL-2 was supplemented at 300 international units (IU) per 1 ml of culture volume every 2 days.

### Flow Cytometry Staining to Determine Transduction Efficiency:

Once Tregs had sufficiently expanded, cells were taken for staining to check phenotype and transduction efficiency. Cells were washed with FACS buffer (phosphate buffer saline (PBS) with 2% fetal bovine serum (FBS) and 2 mM EDTA). Cells were washed in PBS before LIVE/DEAD™ Fixable Near-IR (ThermoFisher Scientific) was used to stain the dead cells for 15 mins at RT. Cells were washed in FACS buffer before Fc blocking with Human TruStain FcXTM (Biolegend) and stained with cell surface antibodies: Brilliant Violet 510™ anti-human CD4 (A161A1; Biolegend, California, USA), PE/Cyanine7 anti-human CD25 (BC96; Biolegend). Washed cells were then fixed and permeabilized using the eBioscience™ FoxP3/Transcription Factor Staining Buffer Set before addition of Alexa Fluor® 488 anti-GFP (FM264G; Biolegend) and PE anti-human FoxP3 antibodies (206D; Biolegend). Cells were acquired by flow cytometry and data was analysed on FlowJo_V10 software.
pSTAT5 Assay Setup for the SupT1 Cells:
The experimental set-up is shown in FIG. 4. SupT1s transduced with the constructs pQTX-0353 (SEQ ID NO: 99) and pQTX-0354 (SEQ ID NO: 100) at M015 alongside mock cells were seeded in a round-bottomed 96 well plate. Cells were treated with Rapamycin at concentrations of, 0.1 nM, 1 nM, 10 nM, 100 nM or 1000 nM on day 0 (48 hour timepoint), then at 24 hours, then at 4 hours pre-staining. All cells were then harvested and stained for pSTAT5 expression.
pSTAT5 Assay Set Up for Human Tregs
Setup (as shown in FIG. 6):
Tregs were rested for 24-36 hours prior to setting up the assay. This assay was setup in U bottom 96 well plates with 150,000-200,000 cell per cells per well. Cells were treated with serial dilutions of Rapamycin (1, 10, 100, 1000, and 10000 nMol), with 0nMol Rapamycin acting as a negative control. Cells treated with 100 IU/ml and 1000 IU/ml IL-2 for 30 min period to staining were used as a positive control for the pSTAT5 detection. All cultures were performed in X-VIVO™ 15 media (Lonza) supplemented with 5% heat-inactivated human serum (Merck Life Science UK Limited) and incubated at 37° C. and 5% CO₂. Readouts were taken at 4 hr and 24 hr post Rapamycin treatment. Subsequently, the cells were stained as detailed below:
pSTAT5 Staining:
Cells were washed in PBS and the supernatant was discarded. Cells were then incubated in 50 ul Live/Dead—Near IF (ThermoFisher) and Human TruStain FcX: Fc receptor blocking solution (Biolegend), in PBS respectively. Cells were washed in FACS Buffer and the supernatant was discarded. 50p1 of surface staining panel containing 0.5 ul of both CD4− BV510 (Biolegend) and CD25− PE-cy7 (Biolegend) in FACS buffer (PBS+2% FBS) was then added and samples were incubated in the fridge. 100 ul of FACS buffer was added per well to wash cells and the supernatant was discarded. 125 ul of Buffer 1 from the PerFix EXPOSE: Phospho-Epitopes Exposure kit (Beckman Coulter) diluted in FBS at a 1:4 ratio (25 ul Buffer 1: 100 ul FBS) was added and samples were incubated before centrifuging and discarding supernatents. 100 ul of Buffer 2 from the PerFix EXPOSE: Phospho-Epitopes Exposure kit (Beckman Coulter) was diluted in FBS at a 1:1 ratio (50 ul Buffer 1: 50 ul FBS)and added to the wells. Samples were again incubated, before centrifuging and discarding the supernatant.

50 ul of intracellular staining panel containing 1 ul of GFP- AF488 (Biolegend) and 2.5 ul pSTAT5-PE (Biolegend) in Buffer 3 from the PerFix EXPOSE: Phospho-Epitopes Exposure kit (Beckman Coulter) was added and incubated, followed by 150 ul of 1× Buffer 4 (diluted 1:20 in H₂O) from the PerFix EXPOSE: Phospho-Epitopes Exposure kit (Beckman Coulter). Samples were centrifuged and supernatants discarded. All samples were resuspended in 200 ul of 1× Buffer 4 (diluted 1:20 in H₂O) from the PerFix EXPOSE: Phospho-Epitopes Exposure kit (Beckman Coulter), before analysing samples by flow cytometry.

### Results

pSTAT5 Assays Using SupT1 Cells
The results shown in FIG. 5 demonstrate that cells containing either pQTX-0353 (SEQ ID NO: 99) or pQTX-0354 (SEQ ID NO: 100) show a significantly higher amount of pSTAT5 after treatment with rapamycin than transduced cells in the absence of rapamycin. After 24 hours of rapamycin treatment at both 10 and 100 nM, pSTAT5 measurements of 67.3% were seen as opposed to 0.63% in the absence of rapamycin. Both constructs are therefore capable of increasing pSTAT5 levels after rapamycin treatment, showing that constructs which comprise a heterologous dimerization domain, that have a transmembrane domain and a JAK1/2 binding domain and a STAT5 association motif can function to provide essential pSTAT5 signaling within cells.
pSTAT5 Assay Using Treg Cells
The data shown in FIG. 7 shows that an increased level of pSTAT5 can also be seen in Tregs comprising constructs encompassed by the invention (pQTX0353 SEQ ID NO: 99) and pQTX0354 (SEQ ID NO: 100)). After 48 hours of treatment with rapamycin, measurements of 16.3% and 15.9% were obtained for cells transduced with pQTX-0353 (SEQ ID NO: 99) and pQTX-0354 (SEQ ID NO: 100), respectively, as compared to only 2.81 and 2.2% of transduced cells in the absence of rapamycin, representing a 5.8-7 fold increase in pSTAT5.

The data shows that transmembrane constructs comprising heterodimerization domains and an endodomain having JAK1/2 binding domains and pSTAT5 association motifs are capable of increasing pSTAT5 within both SupT1s and Treg cells.

1. A chimeric protein capable of providing an inducible STAT5-mediated signal in a cell expressing said protein, said protein being a membrane-associated protein comprising, linked together in any order:
(i) a first heterodimerization domain Ht1;
(ii) a second heterodimerization domain Ht2;
(iii) a signalling domain comprising a STAT5 association motif and a JAK1 and/or JAK2 binding motif, and optionally a JAK3 binding motif;
wherein, in the presence of a chemical inducer of dimerization (CID), an identical pair of the chimeric proteins interact such that Ht1 from one chimeric protein heterodimerizes with Ht2 from the other chimeric protein, causing dimerization of the two signalling domains, such that upon said dimerization said STAT5-mediated signal is induced.

2. The chimeric protein of claim 1, wherein said chimeric protein comprises a transmembrane (TM) domain.

3. The chimeric protein of claim 2, wherein said chimeric protein comprises an extracellular (Exo) domain.

4. The chimeric protein of claim 3, wherein said extracellular domain comprises a safety switch.

5. The chimeric protein of any one of claims 1 to 4, wherein the chimeric protein has the formula:
(i) Ht1-Ht2-[Endo]
(ii) [Endo]-Ht1-Ht2
(iii) Ht1-[Endo]- Ht2
(iv) TM-Ht1-Ht2—[Endo]
(v) TM-[Endo]-Ht1-Ht2
(vi) TM-Ht1- [Endo]-Ht2
(vii) Exo-TM-Ht1-Ht2-[Endo]
(viii) Exo-TM-[Endo]-Ht1-Ht2
(ix) Exo-TM-Ht1-[Endo]-Ht2
(x) Exo-Ht1-Ht2 —TM-[Endo]
(xi) Ht1-Ht2 —TM-[Endo];
wherein [Endo] is the signalling domain, Exo is an extracellular domain, and TM is a transmembrane domain.

6. The chimeric protein of any one of claims 1 to 5, wherein one of Ht1 and Ht2 comprises a FK506-bindng protein (FKBP) and the other comprises a FRB domain of mTOR (FRB).

7. The chimeric protein of claim 6, wherein Ht1 comprises FKBP and Ht2 comprises FRB.

8. The chimeric protein of any one of claims 6 to 7, wherein the CID is rapamycin or a rapamycin analogue.

9. The chimeric protein of any one of claims 1 to 5, wherein:
one of Ht1 and Ht2 comprises a FKBP and the other comprises calcineurin or an FK506-binding fragment thereof, and the CID is FK506;
(ii) one of Ht1 and Ht2 comprises a cyclophilin (CyP) or a cyclosporine (CsA)-binding fragment thereof and the other comprises calcineurin or a CsA-binding fragment thereof, and the CID is CsA;
(iii) one of Ht1 and Ht2 comprises a cyclophilin (CyP) or a cyclosporine (CsA)-binding fragment thereof and the other comprises FKBP, and the CID is FKCsA;
(iv) one of Ht1 and Ht2 comprises a 14-3-3 protein or a fusicoccin-binding fragment thereof and the other comprises a C-terminal peptide of a plant plasma membrane H+-ATPase (PMA), and the CID is fusicoccin;
(v) one of Ht1 and Ht2 comprises a SNAP-tag and the other comprises a HALO-tag, and the CID is a HaXS molecule.

10. The chimeric protein of any one of claims 1 to 9, wherein in the signalling domain the JAK1 and/or JAK2 motif is located N-terminal to the STAT5 motif.

11. The chimeric protein of any one of claims 1 to 10, wherein the signalling domain comprises, N to C-terminal:
35 (i) JAK1 and/or JAK2 motif-STAT5 motif;
(ii) JAK1 and/or JAK2 motif-STAT5 motif-JAK3 motif;
(iii) JAK1 motif-STAT5 motif; or
(iv) JAK1 motif-STAT5 motif-JAK3 motif;
wherein the motifs are linked directly or indirectly to one another, particularly
wherein the JAK3 motif is in reverse orientation.

12. The chimeric protein of any one of claims 1 to 11, wherein:
(i) where the chimeric protein comprises (a) a TM domain or (b) a TM domain and an Exo domain, the JAK1 and/or JAK2 motifs in the signalling domain are linked N-terminal to the STAT5 motif;
(ii) where the signalling domain of the chimeric protein comprises a JAK3 motif, this is linked to the other end of the STAT5 motif than the JAK1 and/or JAK2 motif, particularly in reverse orientation.

13. The chimeric protein of any one of claims 1 to 12, wherein any two domains are linked together by a linker, optionally wherein
(i) Ht1 and Ht2 are linked together by a linker, and the combined domain comprising Ht1 and Ht2 is linked to the signalling domain by a linker; or
(ii) Ht1 and Ht2 are each separately linked to an end of the signalling domain by a linker.

14. The chimeric protein of claim 13, wherein the linker:
is a flexible linker; and/or
(ii) comprises at least one Gly-Ser domain composed solely of Ser and Gly residues; and/or
(iii) comprises at least one Gly-Ser domain having the formula:
25 (S)q-[(G)m-(S)m]n-(G)p
wherein q is 0 or 1; m is an integer from 1-8; n is an integer of at least 1 (e.g. from 1 to 8, or preferably 1 to 6); and p is 0 or an integer from 1 to 3.

15. The chimeric protein of any one of claims 1 to 14, wherein the STAT5 association motif:
(i) comprises an amino acid sequence as set out in any one of SEQ ID NO: 9-15 or a variant which is at least 80, 85, 90, 95, 96, 97, 98 or 99% identical to SEQ ID NO: 9-15; and/or
(ii) comprises the amino acid motif YXXF/L (SEQ ID NO: 16); wherein X is any amino acid; and/or comprises the amino acid motif YCTF (SEQ ID NO: 17), YFFF (SEQ ID NO: 18), YLSL (SEQ ID NO: 19), or YLSLQ (SEQ ID NO: 20).

16. The chimeric protein of any one of claims 1 to 15, wherein:
the JAK1 binding motif comprises an amino acid motif as shown in any one of SEQ ID NO: 21-27 or a variant thereof which is capable of binding JAK1; and/or
(ii) the JAK2 binding motif comprises an amino acid motif as shown in any one of SEQ ID NO: 28-30 or a variant thereof which is capable of binding JAK1; and/or
(iii) the JAK3 binding motif comprises an amino acid motif as shown in any one of SEQ ID NO: 33 or 34 or a variant thereof which is capable of binding JAK1.

17. The chimeric protein of any one of claims 1 to 16, wherein the signalling domain comprises an IL2Rβ sequence selected from an amino acid sequence as set out in any one of SEQ ID NO: 80, 31, or 32, or a sequence which has at least 80% sequence identity to any one of SEQ ID NO: 80, 31, or 32.

18. A nucleic acid molecule comprising a nucleotide sequence encoding a chimeric protein as defined in any one of claims 1 to 17.

19. A construct comprising a nucleic acid molecule as defined in claim 18, and one or more further nucleotide sequences.

20. The construct of claim 19, wherein a further nucleotide sequence:
(i) is a regulatory sequence; and/or
(ii) encodes a protein of interest; and/or
wherein the protein of interest is
(iii) a therapeutic protein; or
(iv) an antigen receptor; or
(v) a CAR or TCR; or
(vi) a safety switch polypeptide;
(vii) a FOXP3 polypeptide.

21. The construct of claim 19 or 20, wherein the further nucleotide sequence encodes a mutant calcineurin which is resistant to at least one calcineurin inhibitor, optionally wherein said mutant calcineurin is sensitive to at least one calcineurin inhibitor.

22. The construct of claim 21, wherein said calcineurin mutant is resistant to FK506 and sensitive to CsA or wherein said calcineurin mutant is resistant to CsA and sensitive to FK506.

23. The construct of any one of claims 19 to 22, wherein the nucleotide sequence encoding the chimeric protein and one or more further nucleotide sequences encoding a protein of interest are separated from one another by a co-expression sequence.

24. A vector comprising a nucleic acid molecule or construct of any one of claims 18 to 23.

25. A cell which expresses a chimeric protein as defined in any one of claims 1 to 17, or which comprises a nucleic acid molecule, construct or vector as defined in any one of claims 18 to 24.

26. The cell of claim 25 which co-expresses the chimeric protein and a protein of interest at its cell surface, particularly wherein the protein of interest is a CAR or TCR.

27. The cell of claim 25 or claim 26 which is a Treg or a precursor therefor.

28. A cell population comprising a cell as defined in any one of claims 25 to 28.

29. A method for making a cell according to any of claims 25 to 28, which comprises the step of introducing into the cell a nucleic acid molecule, construct or vector according to any of claims 18 to 24.

30. A method of promoting the survival or persistence of a cell, said method comprising introducing into the cell, a nucleic acid molecule, construct or vector as defined in any of claims 18 to 24, and exposing the cell to a CID,

31. A method of selectively expanding a cell according to any one of claim 25 or 26, wherein a nucleic acid molecule comprising a mutant calcineurin which is resistant to at least one calcineurin inhibitor has been introduced into the cell, and wherein the cell is cultured in the presence of said calcineurin inhibitor.

32. A pharmaceutical composition comprising a cell or cell population as defined in any one of claims 25 to 28 or a vector as defined in claim 24.

33. A cell or cell population as defined in any one of claims 25 to 28 or a vector as defined in claim 24 or a pharmaceutical composition as defined in claim 32 for use in therapy.

34. A cell of any one of claims 25 to 27, a cell population of claim 28, a vector of claim 24 or a pharmaceutical composition of claim 32 for treating cancer, an infectious, neurodegenerative or inflammatory disease, or for inducing immunosuppression.

35. The cell, cell population, vector or pharmaceutical composition of claim 34, for use in induction of tolerance to a transplant; treating and/or preventing graft-versus-host disease (GvHD), an autoimmune or allergic disease; to promote tissue repair and/or tissue regeneration; or to ameliorate inflammation in a subject, preferably wherein the cell is a Treg cell.

36. A combination product comprising (a) a cell or cell population as defined in any one of claims 25 to 28 or a vector as defined in claim 24 or a pharmaceutical composition as defined in claim 32, and (b) a CID, for use in therapy, preferably ACT or gene therapy.
